SEQUENCE LISTING (10 sequences)

SEQ ID NO: 1 - protein, 25 aa, Homo sapiens
GEHNLSQNDGTEQYVNVQKIVSHPY

SEQ ID NO: 2 - protein, 26 aa, Homo sapiens
CGTEAGRNSWPSQISLQYRSGGSRYH

SEQ ID NO: 3 - protein, 26 aa, Homo sapiens
CRQNWVMTAAHCVDYQKTFRVVAGDH

SEQ ID NO: 4 - protein, 32 aa, Homo sapiens
CVVHPYWNSDNVAAGYDIALLRLAQSVTLNSY

SEQ ID NO: 5 - protein, 26 aa, Homo sapiens
CGKTKTNGQLAQTLQQAYLPSVDYAI

SEQ ID NO: 6 - protein, 25 aa, Homo sapiens
CLVNGKYSVHGVTSFVSSRGCNVSR

SEQ ID NO: 7 - DNA, 20 bp, Artificial Sequence
  Primer, MsCela1 FL1-Forward
ttgtcggagagcacaacctg

SEQ ID NO: 8 - DNA, 20 bp, Artificial Sequence
  Primer, MsCela1 FL1-Reverse
ccaagacaccagcagcattc

SEQ ID NO: 9 - DNA, 20 bp, Artificial Sequence
  Primer, MsGapdh (66-323) mRNA-Forward
agagtgtttcctcgtcccgt

SEQ ID NO: 10 - DNA, 20 bp, Artificial Sequence
  Primer, MsGapdh (66-323) mRNA-Reverse
tgatgttagtggggtctcgc

(19) United States
(12) Patent Application Publication
(10) Pub. No.: US 2023/0002510 A1
(43) Pub. Date: Jan. 5, 2023

(54) CELA-1 INHIBITION FOR TREATMENT OF LUNG DISEASE

(71) Applicant: Children's Hospital Medical Center, Cincinnati, OH (US)

(72) Inventor: Brian Varisco, Cincinnati, OH (US)

(21) Appl. No.: 17/778,188

(22) PCT Filed: Nov. 23, 2020
(86) PCT No.: PCT/US20/61774

§ 371 Date: May 19, 2022

Related U.S. Application Data
(60) Provisional application No. 63/009,134, filed on Apr. 13, 2020.
(60) Provisional application No. 62/940,302, filed on Nov. 26, 2019.

(51) Int. Cl. C07K 16/40; C12N 15/113; A61P 11/00

(52) U.S. Cl. CPC C07K 16/40; C12N 15/1137; A61P 11/00; C07K 2317/21; C07K 2317/622

(57) ABSTRACT

Disclosed herein are compositions and methods for the treatment of one or more progressive lung diseases, which may include, but are not limited to, chronic obstructive pulmonary disease (COPD), emphysema, and AAT deficient lung disease. The compositions useful for the disclosed methods may include an an antigen binding protein such as an anti-CELA1 antibody, an anti-CELA1 scFv, or an anti-CELA1 antisense nucleotide (ASO), which may be administered in an amount sufficient to treat one or more of the aforementioned disease states.

### CROSS REFERENCE TO RELATED APPLICATIONS

This application claims priority to and benefit of US Provisional Application Ser. No. 63/009,134, filed Apr. 13, 2020 and 62/940,302, filed Nov. 26, 2019, the contents of which are incorporated in their entirety for all purposes.

### SEQUENCE LISTING IN ELECTRONIC FORMAT

The present application is being filed along with a Sequence Listing in electronic format. The Sequence Listing is provided as a file entitled CHMC_0738107_CELA1_Inhibition_ST25.txt, created and last saved Nov. 20, 2020, which is 2.83 kilo bytes in size. The information in the electronic format of the Sequence Listing is incorporated herein by reference in its entirety.

### STATEMENT REGARDING FEDERALLY-SPONSORED RESEARCH

This invention was made with government support under A1 Foundation Research Award 498262, NHBLI R01141229, and NHLBI K08HL131261 awarded by the National Institutes of Health. The government has certain rights in the invention.

### BACKGROUND

Lung function is maximal during the 3rd decade of life and slowly declines thereafter. A host of conditions can either reduce the maximal number of alveoli or increase the rate of alveolar loss leading to respiratory insufficiency. Since the 1990s, surfactant replacement therapy and advances in neonatal intensive care has permitted the survival of increasingly premature infants who often have bronchopulmonary dysplasia and reduced peak alveolar number—which many predict will lead to premature respiratory compromise as this population ages.¹ Patients with α-1 antitrypsin (AAT) deficiency presumably have a normal peak number of alveoli but un-opposed proteolytic activity accelerates alveolar loss and leads to emphysema in the 4th and 5th decades of life. A subset of smokers will develop COPD with emphysema being a major element of this disease, and a subset of these patients will experience rapidly progressive emphysema despite smoking cessation.² In these and other clinical situations, the ability to halt or slow progressive airspace destruction could substantially improve respiratory quality of life. Thus, there is a need for compositions and/or methods for addressing one or more of the aforementioned needs in the art.

### BRIEF SUMMARY

Disclosed herein are compositions and methods for the treatment of one or more progressive lung diseases, which may include, but are not limited to, chronic obstructive pulmonary disease (COPD), emphysema, and AAT deficient lung disease. The compositions useful for the disclosed methods may include an anti-CELA1 antigen binding protein (ABP), which may include an anti-CELA1 antibody, and/or an anti-CELA1 scFv, and an anti-CELA1 antisense nucleotide (ASO), any or all of which may be administered in an amount sufficient to treat one or more of the aforementioned disease states.

### BRIEF DESCRIPTION OF THE DRAWINGS

This application file contains at least one drawing executed in color. Copies of this patent or patent application publication with color drawing(s) will be provided by the Office upon request and payment of the necessary fee.

FIG. 1. Cela1 Expression in Murine PPE Model of Emphysema. (A) Lung Cela1 mRNA was significantly increased at 42 and 84 days after tracheal PPE. (B) Proximity ligation in situ hybridization (PLISH) for Cela1 mRNA demonstrated rare expressing cells in PBS-treated lung, (C) small clusters of expressing cells at 3 days post-PPE, and (D) larger numbers of expressing cells concentrated in conducting airways at 21 days post-PPE. (E) Western blot of lung homogenate showed an early increase in high molecular weight Cela1 (previously shown to be Cela1+AAT) and a later 3-fold increase in native Cela1. (F) only this later increase was statistically significant though n=2 per group. *p<0.05, ** p<0.01.

FIG. 2. Cela1 Mediates Late Airspace Destruction in PPE model. (A) Wild type (WT) mice administered tracheal PPE demonstrated airspace destruction which was progressive at (B) 42 and (C) 84 days post-PPE. (D) Cela1−/− mice had a level of emphysema similar to that of WT at 21 days, but at (E) 42 and (F) 84 days, progressive emphysema was absent. (G) Comparison Cela1−/− PBS-treated lung. (H) Mean linear intercept (MLI) quantification of emphysema in WT and Cela1−/− lungs post-PPE showed progression of emphysema at 42 and 84 days in WT but not Cela1−/− lungs treated with PPE. PBS-treated lung MLIs are omitted for clarity but were all ˜50 m and p<0.001 by Kruskal-Wallis and Dunn's post hoc test for each PBS-PPE comparison. The shown comparisons of PPE-treated lung over time Kruskall-Wallis p=0.001, Dunn's post hoc comparison *p<0.05. WT vs. Cela1−/− comparisons by Wilcoxon rank-sum p-values shown.

FIG. 3. Cela1 in Age-Related Airspace Simplification in Mice. (A) The lungs of 70-75 week-old Cela1^{-/-} mice had more soluble tropoelastin than WT lungs. (B) Aged WT mice had less lung elastin with less-dense septal tip bundles than (C) aged Cela1^{-/-} lung demonstrated. (D) Aged Cela1^{-/-} mice did not demonstrate the same degree of simplification as WT mice. (E) Aged WT mice had loss of normal alveolar architecture that was preserved in (F) aged Cela1^{-/-} mice. Central bars represent mean and whiskers standard deviation. *p<0.05, *** p<0.001 by Welch's t-test or ANOVA.

FIG. 4. CELA1 in Human Lung. (A) CELA1 mRNA-positive cells were rare in control adult lung. (B) Some conducting airways in COPD lung had a large number of CELA1-expressing epithelial cells with strongly positive cells in the surrounding tissue. Central bar represents mean and whiskers standard deviation. **p<0.01 by Welch's t-test. (C) COPD lung had greater amounts of native CELA1 protein and tended to have greater amounts of CELA1+AAT compared to control lung. (D) CELA1 mRNA-positive cells were found in a subset of COPD and control conducting airways. A representative COPD airway is shown with CELA1 mRNA labeled green and CELA1 protein red. CELA1 protein staining is light in mRNA-positive cells but more abundant in the underlying matrix. (E) There was substantial variability in the amount of CELA1 mRNA present in control and COPD specimens, but both had more median (black diamond) mRNA than non-COPD smoker lung. p<0.05 by Kruskal-Wallis test.

FIG. 5. CELA1 and Human Lung Elastolytic Activity (A) 30 human lung specimens had quantification of protease and anti-protease mRNAs and quantification of lung elastolytic, gelatinase, and protease activity. Only MMP12 and CELA1 were correlated with lung enzymatic activity, and only CELA1 was statistically significant.

FIG. 6. Anti-CELA1 Antibodies and Human Lung Elastolytic Activity (A) Incubation of human lung homogenates with pre- and post-CELA1 peptide immunization rabbit serum demonstrated that rabbit serum itself inhibited elastase activity in the 15 human lung specimens with the lowest CELA1 mRNA levels “Low” to a lesser extent than the 15 specimens with the highest CELA1 mRNA levels “High.” Polyclonal anti-CELA1 antibody in post-immunization serum neutralized an additional 1% of activity in “Low” specimens and 5% in “High” specimens. (p=0.2). (B) The increased inhibition of lung elastolytic activity was proportionate to the log 10 CELA1 mRNA/18S value. The shaded region is the standard error of the generalized linear model. (C) Four mice were immunized with inactivated CELA1. ELISA titers of all four mice were strong. (D) Splenocytes from these four mice were used to create hybridomas. The twenty strongest ELISA-positive clones by ELISA were tested for ability to inhibit human CELA1 elastolytic activity. Eight clones (red) were selected for validation. (E) Supernatants from these eight clones were tested in triplicate for fractional inhibition of the elastase activity of recombinant human CELA1. Four clones (red) with the highest inhibition were selected. (F) The hybridoma supernatants from these four clones were tested for ability to inhibit lung elastase activity in non-smoker (n=2), smoker (n=4) and COPD (n=6) lung homogenates with the KF4 clone being the most promising. There was insufficient supernatant from the BA8 clone to test all specimens. (G) Serial dilution of the KF4 clone in non-smoker (n=13), smoker (n=9), and COPD (n=6) lung homogenate demonstrated ˜25% elastase inhibition in COPD lung homogenate at a concentration of 695 fM. Kruskal-Wallis p=0.01, *p<0.05 by Dunn's post hoc test.

FIG. 7. CELA1 in Aged Human Lung. (A) Lung CELA1 mRNA levels increased exponentially with age in human lung specimens without clear association with sex or smoking status. Shaded region represents the standard error of the logarithmic model. (B) Western blot for CELA1 in young adult vs aged lung specimens. (C) Quantification of the low (native) and high (CELA1+AAT) molecular weight form of CELA1 shows little change in the amount of native CELA1 protein in aged lung and an overall reduction in the amount of CELA1+AAT in this aged lung. (D) Quantification of low and high molecular weight CELA1 in aged lung specimens. (E) Immunofluorescence image of aged human lung in a region with a high number of CELA1-expressing cells with co-staining for the club cell marker SCGB1A1 showing co-expressing and non-co-expressing cells. Central bar represents mean and whiskers standard deviation. *p<0.05 by Welch's t-test.

FIG. 8. Stretch-Inducible Binding of CELA1 to Healthy Human Lung Tissue. (A) Sectioned, frozen human lung tissue was mounted on a biaxial stretching device and imaged in the presence of elastin in situ zymography substrate and fluorophore-labeled CELA1 and albumin. Un-stretched tissue showed little signal in any of the three channels and was imaged repetitively at the same time interval as stretched lung sections. (B) Stretched lung showed increased binding of CELA1 with stretch but little albumin binding or elastase activity. (C) Control lung had increased binding of CELA1 to lung tissue with stretch. D) Albumin did not increase binding to the lung tissue in response to stretch. (E) Stretch did not induce lung elastase activity. Dashed lines indicates the signal of lung incubated without substrate. *p<0.05 by ANOVA.

FIG. 9. Negative Control Images for PLISH. (A) Cela1^{-/-} lung did not demonstrate any Cela1-mRNA at 21 days post-PPE. (B) A bacterial gene probe did not have any signal in wild type mouse lung at 21 days post-PPE.

FIG. 10. Additional Aged Mouse Lung Data. (A) Western blot of young (8-12 week-old) and aged (70-75 week-old) wild type mouse lung revealed no differences in Cela1 protein levels. (B) Quantitative image analysis of elastin-stained mouse lung issues identified a trend towards increased total lung elastin in aged Cela1^{-/-} mouse lungs. (C) Quantification of lung senescence proteins showed no differences except for a trend towards reduced p53 in aged Cela1^{-/-} mouse lung.

FIG. 11. CELA1 in Human AT2 Cells. CELA1 (red) was present in a subset of AT2 cells in normal (shown here) and COPD human lung. Green signal is staining by HT2-280 which labels the apical membrane of human AT2 cells.

FIG. 12. Aged Human Lung Western Blot. (A) CELA1 and (B) total protein stain of smoker and non-smoker aged human lung.

FIG. 13. 3D-printed Confocal Microscope Lung Stretching Device. (A) Four motors are housed within a plastic case that fits within the microscope mounting plate. A silicone mold (circle) is secured by four clips attached to the motors by Tyvek strips. (B) Image of lung before stretching and after stretching (inset). (C) Quantification of the increase in mold aperture area with number of stretch (steps). The steps used for imaging are shown.

FIG. 14. Determination of KF4 antibody antigen. (A) The five peptides used for mouse immunization (Pep-1, Pep-2, Pep-3, Pep-4, and Pep-5) with the corresponding amino acids indicated (GenBank: EAW58189.1) as well as the peptide used for creating of the CELA1 polyclonal antibody in rabbit (pep-6) and recombinant human Cela1 (MW-35 kDa) were electrophoretically separated and transferred to a PVDF membrane and probed with the KF4 antibody. Bands were detected in the Pep-1 and Pep-2 lanes as well in the recombinant human CELA1 lane. (B) The rabbit polyclonal antibody detected these same bands in Pep-1 and Pep-2 as well as Pep-5 and strong signals with Pep-6 and recombinant human CELA1. (C) ELISA plates coated with Pep-1, Pep-2, or human CELA1 demonstrated no signal for the KF4 antibody with Pep-2 and dilution-dependent reduction in ELISA signal with Pep-1 and human CELA1.

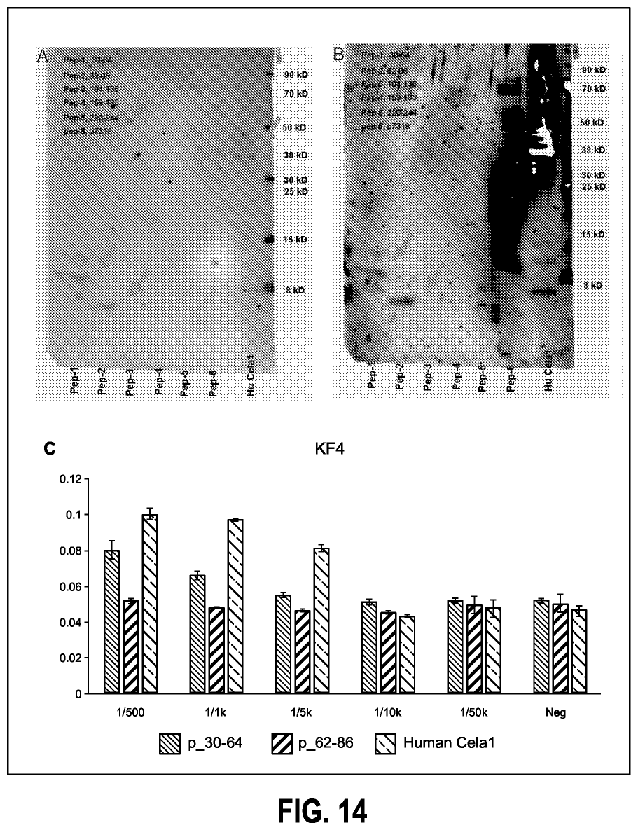

Those of skill in the art will understand that the drawings, described below, are for illustrative purposes only. The drawings are not intended to limit the scope of the present teachings in any way.

### DETAILED DESCRIPTION

### Definitions

Unless otherwise noted, terms are to be understood according to conventional usage by those of ordinary skill in the relevant art. In case of conflict, the present document, including definitions, will control. Preferred methods and materials are described below, although methods and materials similar or equivalent to those described herein may be used in practice or testing of the present invention. All publications, patent applications, patents and other references mentioned herein are incorporated by reference in their entirety. The materials, methods, and examples disclosed herein are illustrative only and not intended to be limiting.

As used herein and in the appended claims, the singular forms “a,” “and,” and “the” include plural referents unless the context clearly dictates otherwise. Thus, for example, reference to “a method” includes a plurality of such methods and reference to “a dose” includes reference to one or more doses and equivalents thereof known to those skilled in the art, and so forth.

The term “about” or “approximately” means within an acceptable error range for the particular value as determined by one of ordinary skill in the art, which will depend in part on how the value is measured or determined, e.g., the limitations of the measurement system. For example, “about” may mean within 1 or more than 1 standard deviation, per the practice in the art. Alternatively, “about” may mean a range of up to 20%, or up to 10%, or up to 5%, or up to 1% of a given value. Alternatively, particularly with respect to biological systems or processes, the term may mean within an order of magnitude, preferably within 5-fold, and more preferably within 2-fold, of a value. Where particular values are described in the application and claims, unless otherwise stated the term “about” meaning within an acceptable error range for the particular value should be assumed.

The term “antigen” refers to a molecule or a portion of a molecule capable of being bound by a selective binding agent, such as an antigen binding protein (including, e.g., an antibody or immunological functional fragment thereof). In some embodiments, the antigen is capable of being used in an animal to produce antibodies capable of binding to that antigen. An antigen can possess one or more epitopes that are capable of interacting with different antigen binding proteins, e.g., antibodies.

As used herein, the term “effective amount” means the amount of one or more active components that is sufficient to show a desired effect. This includes both therapeutic and prophylactic effects. When applied to an individual active ingredient, administered alone, the term refers to that ingredient alone. When applied to a combination, the term refers to combined amounts of the active ingredients that result in the therapeutic effect, whether administered in combination, serially or simultaneously.

The terms “individual,” “host,” “subject,” and “patient” are used interchangeably to refer to an animal that is the object of treatment, observation and/or experiment. Generally, the term refers to a human patient, but the methods and compositions may be equally applicable to non-human subjects such as other mammals. In some embodiments, the terms refer to humans. In further embodiments, the terms may refer to children.

The term “CELA1 activity” includes any biological effect of CELA1.

The terms “polypeptide” or “protein” means a macromolecule having the amino acid sequence of a native protein, that is, a protein produced by a naturally-occurring and non-recombinant cell; or it is produced by a genetically-engineered or recombinant cell, and comprise molecules having the amino acid sequence of the native protein, or molecules having deletions from, additions to, and/or substitutions of one or more amino acids of the native sequence. The term also includes amino acid polymers in which one or more amino acids are chemical analogs of a corresponding naturally-occurring amino acid and polymers. The terms “polypeptide” and “protein” specifically encompass CELA1 antigen binding proteins, antibodies, or sequences that have deletions from, additions to, and/or substitutions of one or more amino acid of antigen-binding protein. The term “polypeptide fragment” refers to a polypeptide that has an amino-terminal deletion, a carboxyl-terminal deletion, and/or an internal deletion as compared with the full-length native protein. Such fragments can also contain modified amino acids as compared with the native protein. In certain embodiments, fragments are about five to 500 amino acids long. For example, fragments can be at least 5, 6, 8, 10, 14, 20, 50, 70, 100, 110, 150, 200, 250, 300, 350, 400, or 450 amino acids long. Useful polypeptide fragments include immunologically functional fragments of antibodies, including binding domains. In the case of a CELA1-binding antibody, useful fragments include but are not limited to a CDR region, a variable domain of a heavy and/or light chain, a portion of an antibody chain or just its variable region including two CDRs, and the like.

“Sequence identity” as used herein indicates a nucleic acid sequence that has the same nucleic acid sequence as a reference sequence, or has a specified percentage of nucleotides that are the same at the corresponding location within a reference sequence when the two sequences are optimally aligned. For example a nucleic acid sequence may have at least 50%, 60%, 70%, 75%, 80%, 85%, 90%, 95%, 96%, 97%, 98%, 99%, or 100% identity to the reference nucleic acid sequence. The length of comparison sequences will generally be at least 5 contiguous nucleotides, preferably at least 10, 11, 12, 13, 14, 15, 16, 17, 18, 19, 20, 21, 22, 23, 24, or 25 contiguous nucleotides, and most preferably the full length nucleotide sequence. Sequence identity may be measured using sequence analysis software on the default setting (e.g., Sequence Analysis Software Package of the Genetics Computer Group, University of Wisconsin Biotechnology Center, 1710 University Avenue, Madison, Wis. 53705). Such software may match similar sequences by assigning degrees of homology to various substitutions, deletions, and other modifications.

The term “therapeutically effective amount” refers to the amount of a CELA1 antigen binding protein determined to produce a therapeutic response in a mammal. Such therapeutically effective amounts are readily ascertained by one of ordinary skill in the art.

Emphysema is an important element of many progressive lung diseases, with chronic obstructive pulmonary disease (COPD) being the most common. With the exception of α 1-antitrypsin (AAT) replacement therapy there are no disease modifying therapies for progressive emphysema. Applicant has previously reported that alveolar type 2 (AT2)-cell synthesized CELA1 is neutralized by AAT and that CELA1 is necessary for emphysema in AAT-deficiency. Here, Applicant has used mouse models and human tissues to show that CELA1 is required for progressive emphysema. In mice, lung injury was induced with tracheal porcine pancreatic elastase. Cela1 began increasing at 21-days, and Cela1^{-/-} mice were protected from continued airspace enlargement at 42 and 84 days (p<0.01). Aged Cela1^{-/-} mice had less airspace simplification than aged WT mice (p<0.05). In humans and mice, CELA1 mRNA and protein were present in subsets of conducing airway epithelial and AT2 cells. COPD lungs had 3-fold more CELA1 protein than control (p<0.05). Among COPD-associated proteases, only CELA1 was positively and significantly correlated with lung elastolytic activity (p<0.001). Rabbit polyclonal and mouse monoclonal anti-CELA1 antibodies inhibited elastolytic activity of CELA1 mRNA-high but not CELA1 mRNA-low human lungs. CELA1 mRNA levels increased exponentially with age, and smoking reduced that ratio of AAT-neutralized:native CELA1 (p<0.05). CELA1 binding to lung tissue increased 6-fold with biaxial strain (p<0.05). It is proposed that CELA1 predisposes to progressive emphysema via (1) increased expression with age, (2) reduced AAT neutralization with smoking, and (3) increased CELA1-binding to lung matrix with strain. Thus, it is believed that anti-CELA1 therapies may provide a novel disease modifying therapy to prevent emphysema progression.

Applicant has found that Chymotrypsin-like Elastase 1 (CELA1) is responsible for progressive airspace destruction in multiple mouse emphysema models, have shown that human lung CELA1 expression and binding to lung matrix are associated with known emphysema risk factors, and have demonstrated that anti-CELA1 antibodies largely inhibit lung elastolytic activity in CELA1 mRNA-high lung specimens.

Disclosed herein are compositions and methods for the treatment of progressive lung disease. In one aspect, the method may comprise administering a CELA1 inhibitor to an individual in need thereof. In one aspect, the progressive lung disease may be chronic obstructive pulmonary disease (COPD), preferably COPD GOLD Stage I or greater. In one aspect, the progressive lung disease may be emphysema. In one aspect, the emphysema may be that of an individual having genetic AAT deficiency. The emphysema may be CT confirmed emphysema. In one aspect, the progressive lung disease may be progressive airspace destruction after injury.

In one aspect, a method of treating a lung disease in a human subject is disclosed. The method may comprise administering a CELA1 inhibitor to a human subject in need thereof. The lung disease may be selected from chronic obstructive pulmonary disease (COPD), optionally COPD GOLD Stage I or greater, emphysema, optionally wherein said emphysema is in an individual having genetic AAT deficiency, optionally wherein said emphysema is CT confirmed emphysema, optionally wherein said emphysema is progressive emphysema, progressive airspace destruction after injury, and combinations thereof.

In one aspect, the CELA1 inhibitor may be an antigen binding protein, or “ABP”. An “antigen binding protein” (“ABP”) as used herein means any protein that binds a specified target antigen. In one aspect, the specified target antigen is a CELA 1 protein or fragment thereof. “Antigen binding protein” may include, for example, antibodies and binding parts thereof, such as immunologically functional fragments. Peptibodies are another example of antigen binding proteins. The term “immunologically functional fragment” (or simply “fragment”) of an antibody or immunoglobulin chain (heavy or light chain) antigen binding protein, as used herein, is a species of antigen binding protein comprising a portion (regardless of how that portion is obtained or synthesized) of an antibody that lacks at least some of the amino acids present in a full-length chain but which is still capable of specifically binding to an antigen. Such fragments are biologically active in that they bind to the target antigen and can compete with other antigen binding proteins, including intact antibodies, for binding to a given epitope. In some embodiments, the fragments are neutralizing fragments. In some embodiments, the fragments can block or reduce the likelihood of the interaction between CELA1 and a target. In one aspect, such a fragment will retain at least one CDR present in the full-length light or heavy chain, and in some embodiments will comprise a single heavy chain and/or light chain or portion thereof. These biologically active fragments may be produced by recombinant DNA techniques or can be produced by enzymatic or chemical cleavage of antigen binding proteins, including intact antibodies Immunologically functional immunoglobulin fragments include, but are not limited to, Fab, a diabody (heavy chain variable domain on the same polypeptide as a light chain variable domain, connected via a short peptide linker that is too short to permit pairing between the two domains on the same chain), Fab′, F(ab′)2, Fv, domain antibodies and single-chain antibodies, and can be derived from a mammalian source, including, for example, human, mouse, rat, or rabbit. It is further contemplated that a functional portion of the antigen binding proteins disclosed herein, for example, one or more CDRs, may be covalently bound to a second protein or to a small molecule to create a therapeutic agent directed to a particular target in the body, possessing bifunctional therapeutic properties, or having a prolonged serum half-life, and may include nonprotein components.

Antigen binding proteins may include antibodies or ABPs derived from antibodies. In certain embodiments, the polypeptide structure of the antigen binding proteins may be based on antibodies, including, but not limited to, monoclonal antibodies, bispecific antibodies, minibodies, domain antibodies, synthetic antibodies (sometimes referred to herein as “antibody mimetics”), chimeric antibodies, humanized antibodies, human antibodies, antibody fusions (sometimes referred to herein as “antibody conjugates”), and fragments thereof, respectively. In some embodiments, the ABP may comprise or consist of avimers (tightly binding peptide).

An antigen binding protein, in one aspect, may be said to “specifically bind” its target antigen when the dissociation constant (Kd) is less than or equal to 10⁻⁷ M. The ABP specifically binds antigen with “high affinity” when the Kd is less than or equal to 5×10⁻⁹ M, and with “very high affinity” when the Kd is less than or equal to 5×10⁻¹⁰ M. In one embodiment, the ABP has a Kd of 10⁻⁹ M. In one embodiment, the off-rate is <1×10⁻⁵. In other embodiments, the ABPs will bind to human CELA1 with a Kd of between about 10⁻⁹ M and 10⁻¹³ M, and in yet another embodiment the ABPs will bind with a Kd less than or equal to 5×10⁻¹⁰. As will be appreciated by one of skill in the art, in some embodiments, any or all of the antigen binding fragments can specifically bind to CELA1. An antigen binding protein is considered to be “selective” when it binds to one target more tightly than it binds to a second target.

In one aspect, the CELA1 inhibitor may be an anti-CELA1 antibody. The anti-CELA1 antibody may be characterized in that the antibody inhibits at least 50%, or at least 60%, or at least 70%, or at least 80%, or at least 90%, or about 100%, of human lung elastolytic activity. In one aspect, the anti-CELA1 antibody products may be modified with RGD motifs to enhance targeting to the lung extracellular space.

In one aspect, the CELA1 inhibitor may be an antigen binding protein (ABP) which binds to one or more residues, or at least two residues, or at least three residues, or at least four residues, or at least five residues, or at least six residues, or at least seven residues, or at least eight residues, or at least nine residues, or at least ten residues in a peptide up to the maximum number of residues of a peptide selected from SEQ ID NO: 2, SEQ ID NO: 3, SEQ ID NO: 4, SEQ ID NO: 5, or SEQ ID NO: 6. In such instances, the ABP may be an antibody, or an isolated monoclonal antibody, or a human antibody that is selective for the corresponding peptide. In one aspect, the anti-CELA1 inhibitor may be an antibody comprising an arginine-glycine-aspartic acid (RGD) motif.

In one aspect, the anti-CELA1 inhibitor may inhibit at least 50%, or at least 60%, or at least 70%, or at least 80%, or at least 90%, or about 100%, of human lung elastolytic activity.

In one aspect, a single-chain variable fragment (scFv) is disclosed. The scFV may comprise a variable region of the heavy (VH) of a disclosed antibody and a variable region of the light chain (VL) of a disclosed antibody, wherein said VH and said VL are connected by a linker peptide, and wherein said scFv is specific for at least one region of human CELA1. In certain aspects, the scFv inhibits at least 50%, or at least 60%, or at least 70%, or at least 80%, or at least 90%, or about 100%, of human lung elastolytic activity.

The CELA1 inhibitor may be administered using methods known in the art. For example, the CELA1 inhibitor may be administered intravenously. In one aspect, the CELA1 inhibitor may be administered via a nebulizer. In a further aspect, the administration is selected from daily, every other day, weekly, every other week, every three weeks, or monthly.

In one aspect, a method of treating a disease or condition selected from progressive lung disease, chronic obstructive pulmonary disease (COPD), emphysema, or progressive airspace destruction after injury, comprising administering an antisense oligonucleotide is disclosed. The antisense oligonucleotide (ASO) used may be one that is characterized by the ability to inhibit CELA1. In one aspect, the ASO may inhibit at least 50%, or at least 60%, or at least 70%, or at least 80%, or at least 90%, or about 100%, of human lung elastolytic activity. By “progressive” it is meant a continued loss of gas exchange surface area due to continued alveolar loss.

Pharmaceutical Compositions

In one aspect, a composition comprising a CELA1 inhibitor is disclosed. The CELA1 inhibitor may be an ABP as disclosed herein. In one aspect, the ABP may be an isolated monoclonal antibody that binds to human CELA1. The monoclonal antibody may bind to at least one residue, or at least two residues, or at least 3 residues, or at least 4 residues, or at least 5 residues, or at least 6 residues, or at least 7 residues of, or at least 8 residues of, or at least 9 residues of, or at least 10 residues, or at least 11 residues, or at least 12 residues of, or at least 13 residues, or at least 14 residues of, or at least 15 residues of, or at least 16 residues of, or at least 17 residues of, or at least 18 residues of, or at least 19 residues of, or at least 20 residues of, or at least 21 residues of, or at least 22 residues of, or at least 23 residues of or at least 24 residues of, or at least 25 residues of, or each residue of a sequence selected from SEQ ID NO: 2, SEQ ID NO: 3, SEQ ID NO: 4, SEQ ID NO: 5, or SEQ ID NO: 6. The composition may comprise an isolated human antibody, and may further comprise a carrier that is one or both of sterile and isotonic or more generally in a form suitable for administration to an individual in need thereof. In certain aspects, the isolated monoclonal antibody of the composition may inhibit at least 50%, or at least 60%, or at least 70%, or at least 80%, or at least 90%, or about 100%, of human lung elastolytic activity.

In one aspect, the antibody, ASO, and/or scFv provided herein may be administered in a dosage form selected from intravenous or subcutaneous unit dosage form, oral, parenteral, intravenous, and subcutaneous. In some embodiments, the ABP, antibody, ASO, and/or scFv provided herein may be formulated into liquid preparations. Suitable forms include suspensions, syrups, elixirs, and the like. In some embodiments, unit dosage forms for oral administration include tablets and capsules. Unit dosage forms configured for administration once a day; however, in certain embodiments it may be desirable to configure the unit dosage form for administration twice a day, or more.

In one aspect, pharmaceutical compositions are isotonic with the blood or other body fluid of the recipient. The isotonicity of the compositions may be attained using sodium tartrate, propylene glycol or other inorganic or organic solutes. An example includes sodium chloride. Buffering agents may be employed, such as acetic acid and salts, citric acid and salts, boric acid and salts, and phosphoric acid and salts. Parenteral vehicles include sodium chloride solution, Ringer's dextrose, dextrose and sodium chloride, lactated Ringer's or fixed oils. Intravenous vehicles include fluid and nutrient replenishers, electrolyte replenishers (such as those based on Ringer's dextrose), and the like.

Viscosity of the pharmaceutical compositions may be maintained at the selected level using a pharmaceutically acceptable thickening agent. Methylcellulose is useful because it is readily and economically available and is easy to work with. Other suitable thickening agents include, for example, xanthan gum, carboxymethyl cellulose, hydroxypropyl cellulose, carbomer, and the like. In some embodiments, the concentration of the thickener will depend upon the thickening agent selected. An amount may be used that will achieve the selected viscosity. Viscous compositions are normally prepared from solutions by the addition of such thickening agents.

A pharmaceutically acceptable preservative may be employed to increase the shelf life of the pharmaceutical compositions. Benzyl alcohol may be suitable, although a variety of preservatives including, for example, parabens, thimerosal, chlorobutanol, or benzalkonium chloride may also be employed. A suitable concentration of the preservative is typically from about 0.02% to about 2% based on the total weight of the composition, although larger or smaller amounts may be desirable depending upon the agent selected. Reducing agents, as described above, may be advantageously used to maintain good shelf life of the formulation.

In one aspect, the antibody, ASO, and/or scFv provided herein may be in admixture with a suitable carrier, diluent, or excipient such as sterile water, physiological saline, glucose, or the like, and may contain auxiliary substances such as wetting or emulsifying agents, pH buffering agents, gelling or viscosity enhancing additives, preservatives, flavoring agents, colors, and the like, depending upon the route of administration and the preparation desired. See, e.g., “Remington: The Science and Practice of Pharmacy”, Lippincott Williams & Wilkins; 20th edition (Jun. 1, 2003) and “Remington's Pharmaceutical Sciences,” Mack Pub. Co.; 18th and 19th editions (December 1985, and June 1990, respectively). Such preparations may include complexing agents, metal ions, polymeric compounds such as polyacetic acid, polyglycolic acid, hydrogels, dextran, and the like, liposomes, microemulsions, micelles, unilamellar or multilamellar vesicles, erythrocyte ghosts or spheroblasts. Suitable lipids for liposomal formulation include, without limitation, monoglycerides, diglycerides, sulfatides, lysolecithin, phospholipids, saponin, bile acids, and the like. The presence of such additional components may influence the physical state, solubility, stability, rate of in vivo release, and rate of in vivo clearance, and are thus chosen according to the intended application, such that the characteristics of the carrier are tailored to the selected route of administration.

For oral administration, the pharmaceutical compositions may be provided as a tablet, aqueous or oil suspension, dispersible powder or granule, emulsion, hard or soft capsule, syrup or elixir. Compositions intended for oral use may be prepared according to any method known in the art for the manufacture of pharmaceutical compositions and may include one or more of the following agents: sweeteners, flavoring agents, coloring agents and preservatives. Aqueous suspensions may contain the active ingredient in admixture with excipients suitable for the manufacture of aqueous suspensions.

Formulations for oral use may also be provided as hard gelatin capsules, wherein the active ingredient(s) are mixed with an inert solid diluent, such as calcium carbonate, calcium phosphate, or kaolin, or as soft gelatin capsules. In soft capsules, the antibody, ASO, and/or scFv provided herein may be dissolved or suspended in suitable liquids, such as water or an oil medium, such as peanut oil, olive oil, fatty oils, liquid paraffin, or liquid polyethylene glycols. Stabilizers and microspheres formulated for oral administration may also be used. Capsules may include push-fit capsules made of gelatin, as well as soft, sealed capsules made of gelatin and a plasticizer, such as glycerol or sorbitol. The push-fit capsules may contain the active ingredient in admixture with fillers such as lactose, binders such as starches, and/or lubricants, such as talc or magnesium stearate and, optionally, stabilizers.

Tablets may be uncoated or coated by known methods to delay disintegration and absorption in the gastrointestinal tract and thereby provide a sustained action over a longer period of time. For example, a time delay material such as glyceryl monostearate may be used. When administered in solid form, such as tablet form, the solid form typically comprises from about 0.001 wt. % or less to about 50 wt. % or more of active ingredient(s), for example, from about 0.005, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, or 1 wt. % to about 2, 3, 4, 5, 6, 7, 8, 9, 10, 15, 20, 25, 30, 35, 40, or 45 wt. %.

Tablets may contain the active ingredients in admixture with non-toxic pharmaceutically acceptable excipients including inert materials. For example, a tablet may be prepared by compression or molding, optionally, with one or more additional ingredients. Compressed tablets may be prepared by compressing in a suitable machine the active ingredients in a free-flowing form such as powder or granules, optionally mixed with a binder, lubricant, inert diluent, surface active or dispersing agent. Molded tablets may be made by molding, in a suitable machine, a mixture of the powdered active agent moistened with an inert liquid diluent.

In some embodiments, each tablet or capsule contains from about 1 mg or less to about 1,000 mg or more of a active agent provided herein, for example, from about 10, 20, 30, 40, 50, 60, 70, 80, 90, or 100 mg to about 150, 200, 250, 300, 350, 400, 450, 500, 550, 600, 650, 700, 750, 800, or 900 mg. In some embodiments, tablets or capsules are provided in a range of dosages to permit divided dosages to be administered. A dosage appropriate to the patient and the number of doses to be administered daily may thus be conveniently selected. In certain embodiments two or more of the therapeutic agents may be incorporated to be administered into a single tablet or other dosage form (e.g., in a combination therapy); however, in other embodiments the therapeutic agents may be provided in separate dosage forms.

Suitable inert materials include diluents, such as carbohydrates, mannitol, lactose, anhydrous lactose, cellulose, sucrose, modified dextrans, starch, and the like, or inorganic salts such as calcium triphosphate, calcium phosphate, sodium phosphate, calcium carbonate, sodium carbonate, magnesium carbonate, and sodium chloride. Disintegrants or granulating agents may be included in the formulation, for example, starches such as corn starch, alginic acid, sodium starch glycolate, Amberlite, sodium carboxymethylcellulose, ultramylopectin, sodium alginate, gelatin, orange peel, acid carboxymethyl cellulose, natural sponge and bentonite, insoluble cationic exchange resins, powdered gums such as agar, or karaya, or alginic acid or salts thereof.

Binders may be used to form a hard tablet. Binders include materials from natural products such as acacia, starch and gelatin, methyl cellulose, ethyl cellulose, carboxymethyl cellulose, polyvinyl pyrrolidone, hydroxypropylmethyl cellulose, and the like.

Lubricants, such as stearic acid or magnesium or calcium salts thereof, polytetrafluoroethylene, liquid paraffin, vegetable oils and waxes, sodium lauryl sulfate, magnesium lauryl sulfate, polyethylene glycol, starch, talc, pyrogenic silica, hydrated silicoaluminate, and the like, may be included in tablet formulations.

Surfactants may also be employed, for example, anionic detergents such as sodium lauryl sulfate, dioctyl sodium sulfosuccinate and dioctyl sodium sulfonate, cationic such as benzalkonium chloride or benzethonium chloride, or nonionic detergents such as polyoxyethylene hydrogenated castor oil, glycerol monostearate, polysorbates, sucrose fatty acid ester, methyl cellulose, or carboxymethyl cellulose.

Controlled release formulations may be employed wherein the active agent or analog(s) thereof is incorporated into an inert matrix that permits release by either diffusion or leaching mechanisms. Slowly degenerating matrices may also be incorporated into the formulation. Other delivery systems may include timed release, delayed release, or sustained release delivery systems.

Coatings may be used, for example, nonenteric materials such as methyl cellulose, ethyl cellulose, hydroxyethyl cellulose, methylhydroxyethyl cellulose, hydroxypropyl cellulose, hydroxypropyl-methyl cellulose, sodium carboxy-methyl cellulose, providone and the polyethylene glycols, or enteric materials such as phthalic acid esters. Dyestuffs or pigments may be added for identification or to characterize different combinations of active agent doses.

When administered orally in liquid form, a liquid carrier such as water, petroleum, oils of animal or plant origin such as peanut oil, mineral oil, soybean oil, or sesame oil, or synthetic oils may be added to the active ingredient(s). Physiological saline solution, dextrose, or other saccharide solution, or glycols such as ethylene glycol, propylene glycol, or polyethylene glycol are also suitable liquid carriers. The pharmaceutical compositions may also be in the form of oil-in-water emulsions. The oily phase may be a vegetable oil, such as olive or arachis oil, a mineral oil such as liquid paraffin, or a mixture thereof. Suitable emulsifying agents include naturally-occurring gums such as gum acacia and gum tragamayth, naturally occurring phosphatides, such as soybean lecithin, esters or partial esters derived from fatty acids and hexitol anhydrides, such as sorbitan mono-oleate, and condensation products of these partial esters with ethylene oxide, such as polyoxyethylene sorbitan mono-oleate. The emulsions may also contain sweetening and flavoring agents.

Pulmonary delivery of the active agent may also be employed. The active agent may be delivered to the lungs while inhaling and traverses across the lung epithelial lining to the blood stream. A wide range of mechanical devices designed for pulmonary delivery of therapeutic products may be employed, including but not limited to nebulizers, metered dose inhalers, and powder inhalers, all of which are familiar to those skilled in the art. These devices employ formulations suitable for the dispensing of active agent. Typically, each formulation is specific to the type of device employed and may involve the use of an appropriate propellant material, in addition to diluents, adjuvants, and/or carriers useful in therapy.

The active ingredients may be prepared for pulmonary delivery in particulate form with an average particle size of from 0.1 um or less to 10 um or more, for example, from about 0.2, 0.3, 0.4, 0.5, 0.6, 0.7, 0.8, or 0.9 μm to about 1.0, 1.5, 2.0, 2.5, 3.0, 3.5, 4.0, 4.5, 5.0, 5.5, 6.0, 6.5, 7.0, 7.5, 8.0, 8.5, 9.0, or 9.5 μm. Pharmaceutically acceptable carriers for pulmonary delivery of active agent include carbohydrates such as trehalose, mannitol, xylitol, sucrose, lactose, and sorbitol. Other ingredients for use in formulations may include DPPC, DOPE, DSPC, and DOPC. Natural or synthetic surfactants may be used, including polyethylene glycol and dextrans, such as cyclodextran. Bile salts and other related enhancers, as well as cellulose and cellulose derivatives, and amino acids may also be used. Liposomes, microcapsules, microspheres, inclusion complexes, and other types of carriers may also be employed.

Pharmaceutical formulations suitable for use with a nebulizer, either jet or ultrasonic, typically comprise the active agent dissolved or suspended in water at a concentration of about 0.01 or less to 100 mg or more of active agent per mL of solution, for example, from about 0.1, 1, 2, 3, 4, 5, 6, 7, 8, 9, or 10 mg to about 15, 20, 25, 30, 35, 40, 45, 50, 55, 60, 65, 70, 75, 80, 85, or 90 mg per mL of solution. The formulation may also include a buffer and a simple sugar (e.g., for protein stabilization and regulation of osmotic pressure). The nebulizer formulation may also contain a surfactant, to reduce or prevent surface induced aggregation of the active agent caused by atomization of the solution in forming the aerosol.

Formulations for use with a metered-dose inhaler device generally comprise a finely divided powder containing the active ingredients suspended in a propellant with the aid of a surfactant. The propellant may include conventional propellants, such as chlorofluorocarbons, hydrochlorofluorocarbons, hydrofluorocarbons, and hydrocarbons. Example propellants include trichlorofluoromethane, dichlorodifluoromethane, dichlorotetrafluoroethanol, 1,1,1,2-tetrafluoroethane, and combinations thereof. Suitable surfactants include sorbitan trioleate, soya lecithin, and oleic acid.

Formulations for dispensing from a powder inhaler device typically comprise a finely divided dry powder containing active agent, optionally including a bulking agent, such as lactose, sorbitol, sucrose, mannitol, trehalose, or xylitol in an amount that facilitates dispersal of the powder from the device, typically from about 1 wt. % or less to 99 wt. % or more of the formulation, for example, from about 5, 10, 15, 20, 25, 30, 35, 40, 45, or 50 wt. % to about 55, 60, 65, 70, 75, 80, 85, or 90 wt. % of the formulation.

In some embodiments, an active agent provided herein may be administered by intravenous, parenteral, or other injection, in the form of a pyrogen-free, parenterally acceptable aqueous solution or oleaginous suspension. Suspensions may be formulated according to methods well known in the art using suitable dispersing or wetting agents and suspending agents. The preparation of acceptable aqueous solutions with suitable pH, isotonicity, stability, and the like, is within the skill in the art. In some embodiments, a pharmaceutical composition for injection may include an isotonic vehicle such as 1,3-butanediol, water, isotonic sodium chloride solution, Ringer's solution, dextrose solution, dextrose and sodium chloride solution, lactated Ringer's solution, or other vehicles as are known in the art. In addition, sterile fixed oils may be employed conventionally as a solvent or suspending medium. For this purpose, any bland fixed oil may be employed including synthetic mono or diglycerides. In addition, fatty acids such as oleic acid may likewise be used in the formation of injectable preparations. The pharmaceutical compositions may also contain stabilizers, preservatives, buffers, antioxidants, or other additives known to those of skill in the art.

The duration of the injection may be adjusted depending upon various factors, and may comprise a single injection administered over the course of a few seconds or less, to 0.5, 0.1, 0.25, 0.5, 0.75, 1, 2, 3, 4, 5, 6, 7, 8, 9, 10, 11, 12, 13, 14, 15, 16, 17, 18, 19, 20, 21, 22, 23, or 24 hours or more of continuous intravenous administration.

In some embodiments, the antibody, ASO, and/or scFv provided herein may additionally employ adjunct components conventionally found in pharmaceutical compositions in their art-established fashion and at their art-established levels.

In some embodiments, the antibody, ASO, and/or scFv provided herein may be provided to an administering physician or other health care professional in the form of a kit. The kit is a package which houses a container which contains the active agent(s) in a suitable pharmaceutical composition, and instructions for administering the pharmaceutical composition to a subject. The kit may optionally also contain one or more additional therapeutic agents currently employed for treating a disease state as described herein. For example, a kit containing one or more compositions comprising the antibody, ASO, and/or scFv provided herein in combination with one or more additional active agents may be provided, or separate pharmaceutical compositions containing an active agent as provided herein and additional therapeutic agents may be provided. The kit may also contain separate doses of an active agent provided herein for serial or sequential administration. The kit may optionally contain one or more diagnostic tools and instructions for use. The kit may contain suitable delivery devices, e.g., syringes, and the like, along with instructions for administering the active agent(s) and any other therapeutic agent. The kit may optionally contain instructions for storage, reconstitution (if applicable), and administration of any or all therapeutic agents included. The kits may include a plurality of containers reflecting the number of administrations to be given to a subject.

### EXAMPLES

The following non-limiting examples are provided to further illustrate embodiments of the invention disclosed herein. It should be appreciated by those of skill in the art that the techniques disclosed in the examples that follow represent approaches that have been found to function well in the practice of the invention, and thus may be considered to constitute examples of modes for its practice. However, those of skill in the art should, in light of the present disclosure, appreciate that many changes may be made in the specific embodiments that are disclosed and still obtain a like or similar result without departing from the spirit and scope of the invention.

Lung elastic fibers have a half-life of ˜70 years³ and are key to normal lung function. Elastic fiber destruction is a hallmark of emphysematous disease, and a host of matrix metalloproteinases (MMPs) and serine proteases have been implicated in different emphysematous disorders.⁴ With the possible exception of AAT replacement therapy in AAT-deficient emphysema, targeted protease inhibition strategies in humans have failed.⁵ Applicant tested the role of a novel serine protease, Chymotrypsin-like Elastase 1 (CELA1) in emphysema. Here, Applicant has established that CELA1 is developmentally regulated, reduces postnatal lung compliance and is required for emphysema in a murine model of AAT-deficiency.^{6,7} Applicant has found that, unlike other emphysema-related proteases, CELA1 plays no role in early lung injury, but rather, is believed to be responsible for progressive airspace destruction after injury and in the normal alveolar loss associated with aging. Applicant has found that CELA1 mRNA levels correlate strongly with the proteolytic state of the human lung and that the elastolytic activity of CELA1-high lung can be reduced with anti-CELA1 antibodies. Applicant has further found that CELA1 can account for the age, smoking and preceding-injury risk factors of emphysema in that its mRNA levels increase with age, its neutralization by AAT is reduced with smoking, and its binding to lung matrix is increased with strain.

Late Increase in Cela1 After Lung Injury

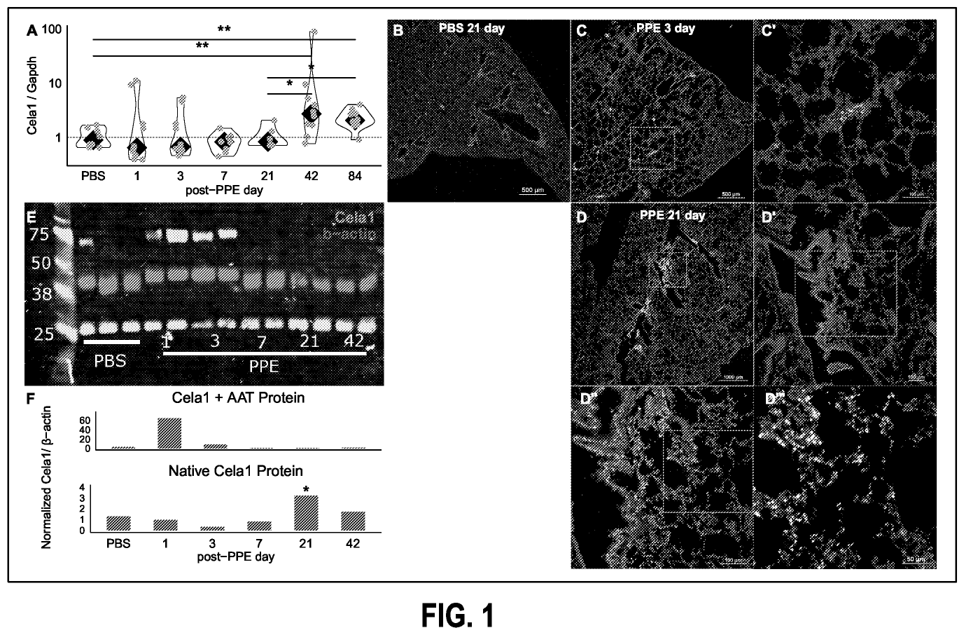

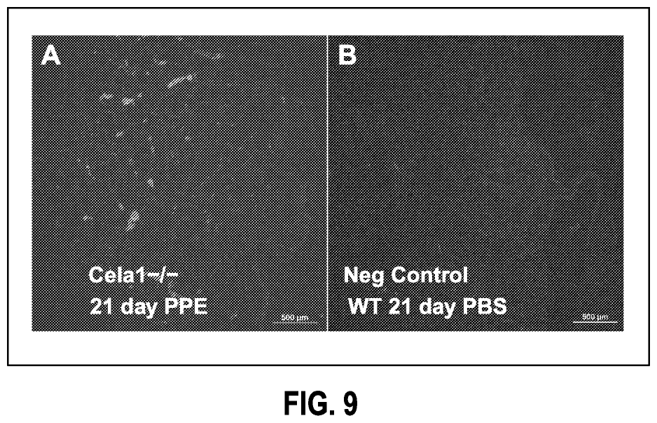

Applicant assessed Cela1 expression in the murine tracheal PPE model of emphysema. Increased Cela1 mRNA was noted in several specimens in the days after PPE, but it was significantly increased at 42 and 84 days post-PPE with median values 2.7 and 2-fold increased respectively (FIG. 1, panel A). Proximity ligation in situ hybridization (PLISH) for Cela1 mRNA demonstrated very few positive cells in PBS-treated lung (FIG. 1, panel B), rare clusters of expressing cells at 3 days post-PPE (FIG. 1, panel C), and substantially increased numbers of Cela1-expressing cells in a subset of conducting airways at 21, 42, and 84 days (FIG. 1, panel D, FIG. 9). Applicant has previously shown that the ˜70 kDa Cela1 species in mouse lung was Cela1+AAT protein.⁷ At 1 and 3 days post-PPE Cela1+AAT protein was elevated, perhaps related to leak of serum proteins into airspaces. At 21 and 42 days, native Cela1 protein levels were increased ˜3-fold (FIG. 1, panels E&F). Overall, these data show that Cela1 expression begins increasing between 3 and 6 weeks following lung injury with localization to some but not all of the conducting airways.

Cela1 Mediates Emphysema Progression in Mouse PPE Model

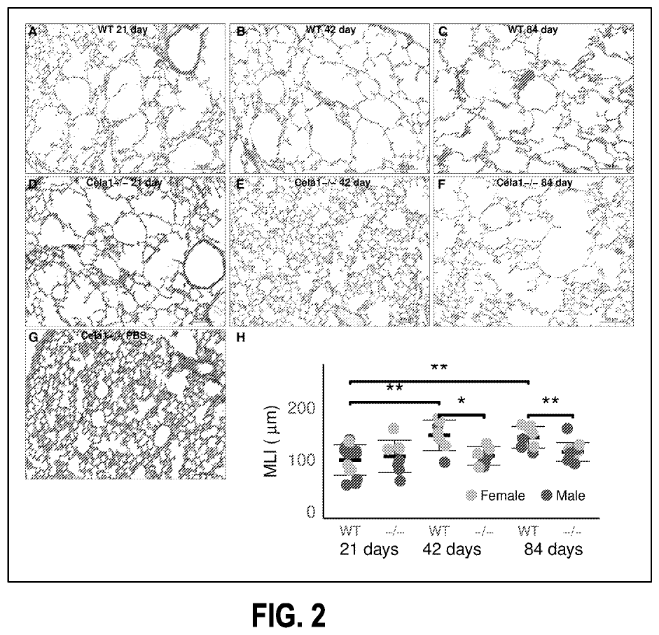

Applicant assessed emphysema at 21, 42, and 84 days post-PPE in WT and Cela1^{-/-} mice. Cela1^{-/-} and WT mice had the same degree of airspace destruction up until 21 days, but Cela1^{-/-} mice were protected from the progressive airspace destruction observed in WT mice at 42 and 84 days. Consistent with reports of increased susceptibility of females to emphysema (8-10) female mice tended to have worsened emphysema, though this was not statistically significant (FIG. 2). These data show that in this murine model of emphysema, Cela1 does not play an important role in early airspace injury, but Cela1 is necessary for the emphysema progression that occurs after 21 days.

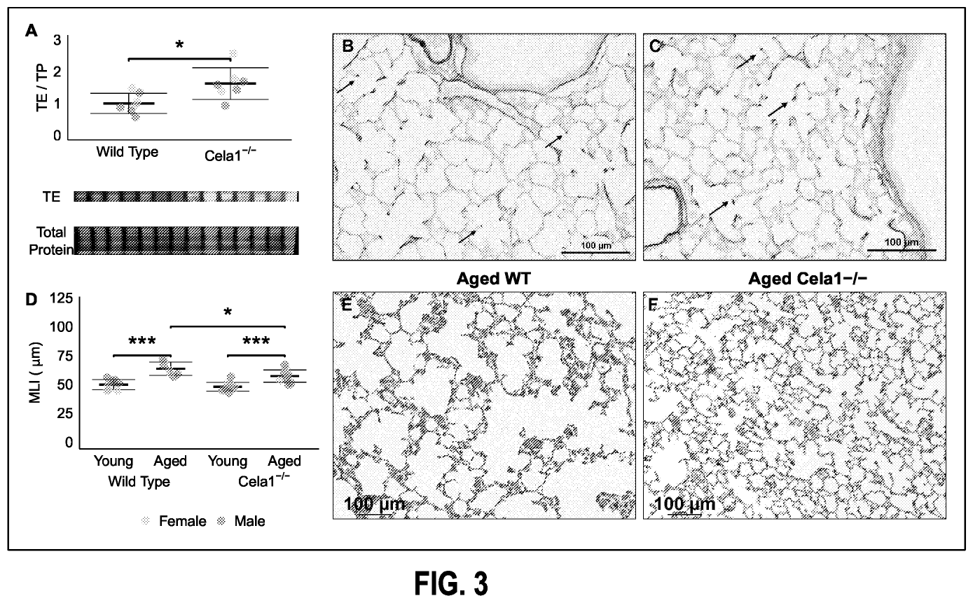

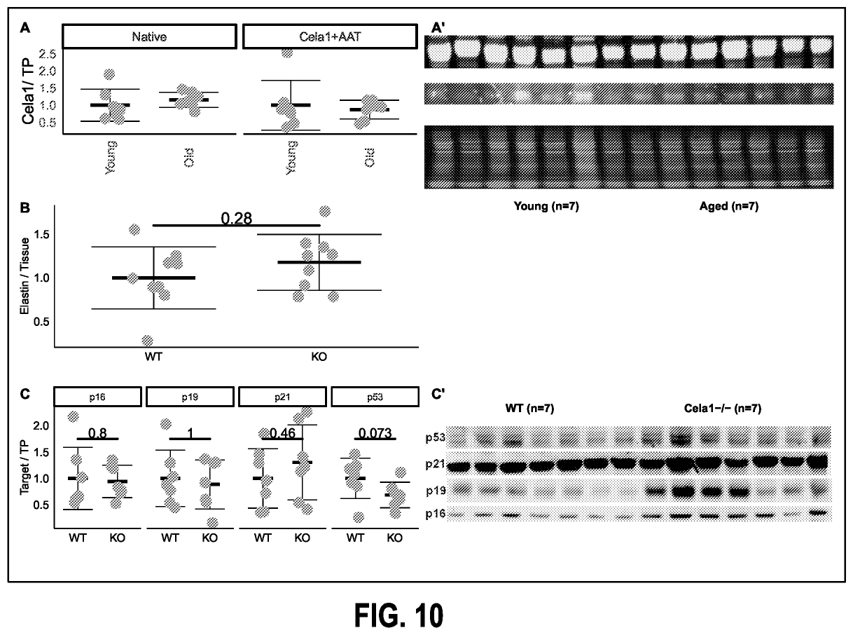

Cela1 in Murine Age-related Airspace Simplification. A hallmark of aging in humans and in mice is alveolar loss and progressive airspace simplification.⁸ Since Cela1 appeared to be involved in late lung remodeling, Applicant evaluated the lungs of aged WT and Cela1^{-/-} mice. The Cela1 protein levels of young adult (8-10 weeks) and aged mouse lung (70-75 weeks) were no different (FIG. 10); however aged Cela1^{-/-} lung had 45% more soluble tropoelastin (FIG. 3A) and similarly increased insoluble elastin (FIG. 10) with less destruction of elastin architecture (FIG. 3 panels B&C). Cela1^{-/-} mice were largely protected from the age-related airspace simplification seen in WT mice (FIG. 3, panels D-F). As cellular senescence is known to be important in the pathogenesis of emphysema and other lung diseases⁹ Applicant evaluated protein levels of p16/p19, p21, and p53. Applicant found no significant differences in the whole lung levels of these factors (FIG. 10). These data implicate CELA1 in the lung remodeling processes of aging.

CELA1 in Human Emphysema

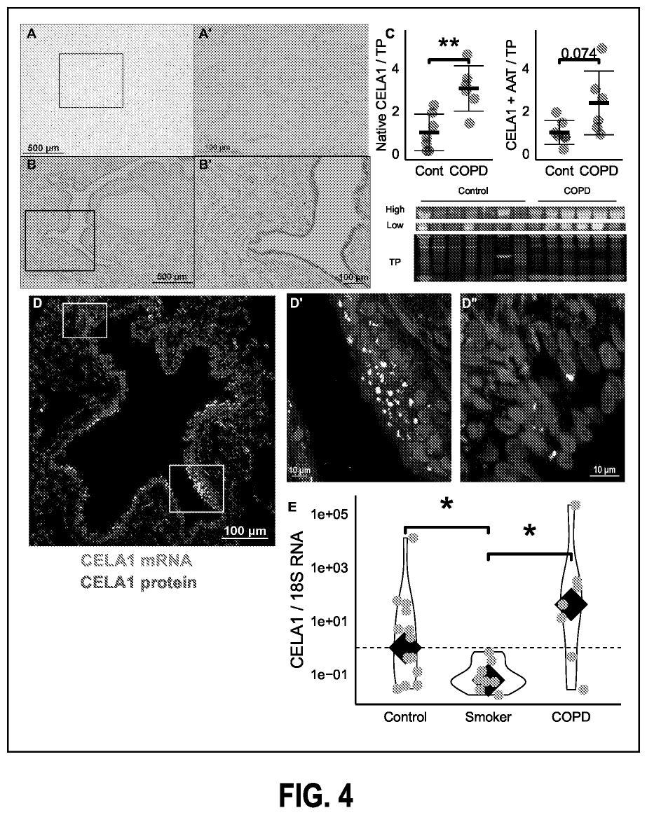

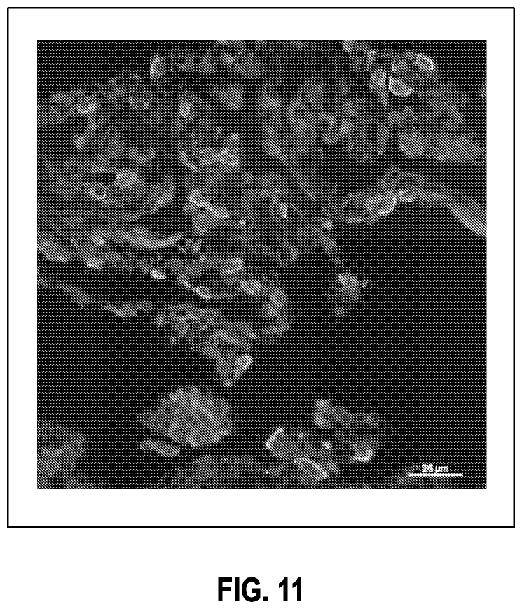

Applicant previously demonstrated the presence of CELA1 mRNA in human lung and that a high molecular weight species of CELA1 (˜70 kDa) was a covalent complex of Cela1 and AAT. To further evaluate CELA1 in COPD, Applicant first performed immunohistochemistry. Counting of CELA1-protein-positive cells in tile scan sections of COPD and control lung section showed 97 vs 5 CELA1-positive cells per mm² respectively (n=3&4 respectively, p=0.01 by Welch's t-test). These cells were largely localized to conducting airways (FIG. 4, panels A&B). Western blot showed increased high and low molecular weight CELA1 species in COPD lung (FIG. 4C). PLISH of non-lung organ donor lung and COPD lung demonstrated scattered regions of CELA1 mRNA in the lung periphery, but subsets of conducting airways with CELA1 mRNA in epithelial cells and CELA1 protein both in these cells and in the underlying matrix (FIG. 4D) Immunohistochemistry showed that peripheral CELA1-expressing cells were alveolar type 2 (AT2) cells (FIG. 11), as Applicant demonstrated in AAT-deficient emphysema.⁷ The CELA1-mRNA levels of COPD specimens were greater than smokers, but some control lung specimens had elevated CELA1 mRNA levels as well (FIG. 4, panel F). These protein and mRNA data show that CELA1 is present in a subset of conducing airways in human lung just as in mice and that there is generally more CELA1 in COPD lung than non-COPD lung. The clinical status of non-smoker organ donor lung makes comparison between control and COPD difficult.

CELA1 Inhibition Reduces Lung Elastolytic Activity

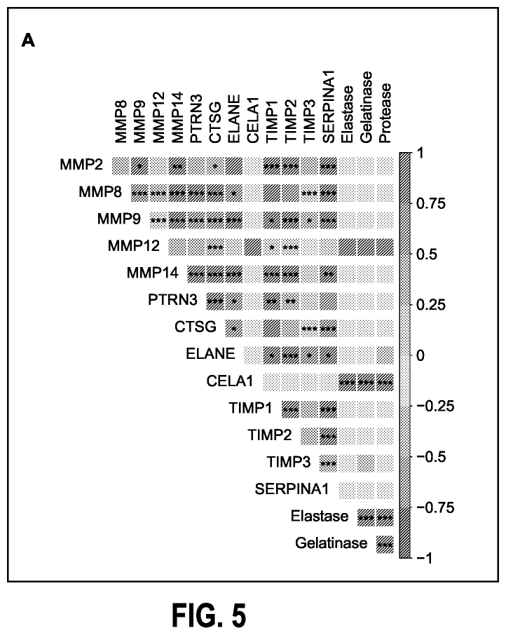

Applicant used lung specimens from 7 COPD subjects, 9 smokers without COPD, and 14 non-lung organ donors to correlate lung elastolytic, gelatinase, and protease activity with mRNA levels of proteases and anti-proteases important in emphysema. CELA1 was the only gene with mRNA levels significantly correlated with lung proteolytic activity (FIG. 5, panel A). Using the median CELA1 mRNA level as a cutoff, CELA1-low and high specimens had 14% and 20% inhibition of elastolytic activity respectively when incubated with rabbit serum with an additional 1% and 5% inhibition when incubated with post-CELA1 immunization rabbit serum (FIG. 5, panel B). Correlating CELA1 expression with antibody-mediated inhibition, specimens with higher levels of CELA1 had greater inhibition of lung elastolytic activity (FIG. 5, panel C). These data show that CELA1 is an important determinant in human lung elastolytic activity and suggest the benefit of anti-CELA1 therapies.

Anti-CELA1 Monoclonal Antibodies

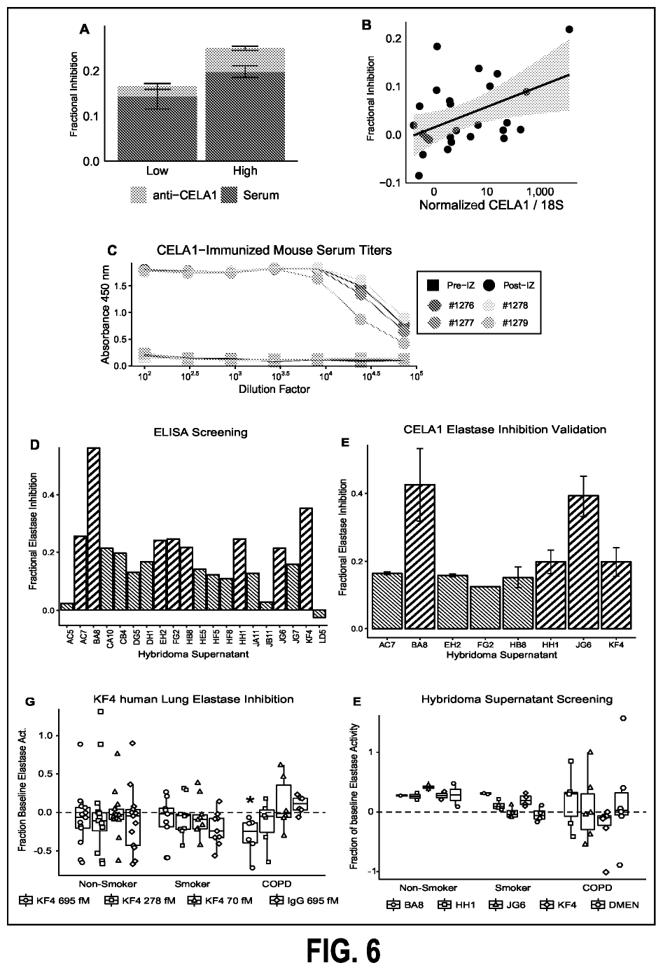

Since rabbit polyclonal antibodies do not represent a viable therapy for human emphysema, applicants developed hybridomas from mice immunized against CELA1 (FIG. 6, panel A) and tested these antibodies as was done for rabbit polyclonal antibodies. Hybridoma screening yielded twenty positive clones which were tested for inhibition of the elastolytic activity of recombinant human CELA1 with validation of the most promising clones (FIG. 6, panels B&C). Hybridomas of the four clones with the greatest inhibition of CELA1 elastolytic activity were expanded and the ability of antibodies purified from the supernatants of these hybridomas tested for ability to inhibit human lung elastolytic activity. All four inhibited between 60 and 100% of lung elastolytic activity in non-smoker, smoker, and COPD lung homogenates (FIG. 6, panel D). These data suggest that antibody inhibition of CELA1 can alter human lung elastolytic activity.

Genomics of Human CELA1

If CELA1 were important in emphysema, Applicant considered why it had not been previously identified in large COPD genomics studies.¹⁰⁻¹² One hundred and fifteen CELA1 variants were identified in Broad Institute,¹³ dbGap,¹⁴ and ClinVar¹⁵ databases, but only five have allele frequencies of >1%, and none of these were predicted loss of function mutations by SIFT and PolyPhen-2.¹⁶ The most common predicted loss-of-function mutation (51735071, AG->A) has an allele frequency of 0.03% with one Caucasian and one Latino homozygote reported. This conservation of CELA1 function is consistent with the invariant conservation of CELA1 in the placental mammal lineage.⁷ Thus, as CELA1 loss-of-function mutations are rare, they would be unlikely to have been identified as protective in population-level genomics studies.

Human Lung CELA1 Increases with Age

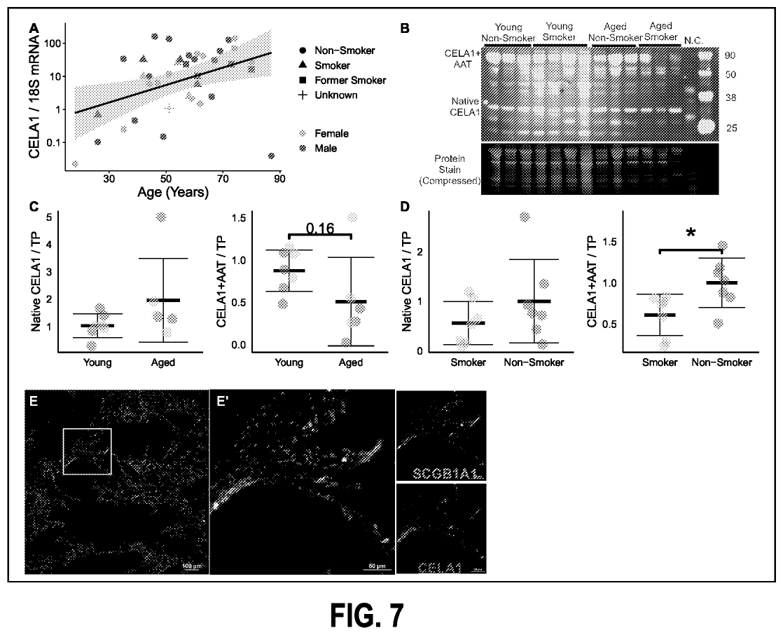

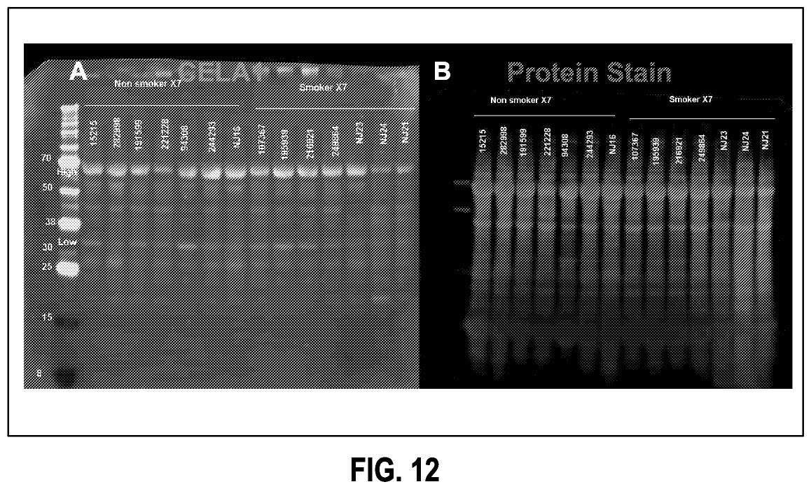

Given data that Cela1 was important in age-related mouse lung remodeling, Applicant assessed CELA1 mRNA and protein levels in aged human lung. Although levels were variable, CELA1 mRNA levels increased exponentially with age and were not associated with sex or smoking status (FIG. 7, panel A). Despite mRNA findings, naive CELA1 protein levels were no different in young and aged lung (FIG. 7, panels B&C), but AAT-bound CELA1 tended to be less in aged lung. Since this difference appeared to be driven by smoking, Applicant compared 7 aged non-smoker lungs to 7 aged smoker lungs. Smoking reduced CELA1+AAT levels in these aged lung specimens. (FIG. 7D, FIG. 12). Immunofluorescence of aged human lung showed regionalization of CELA1 protein with many of these CELA1-positive cells being club cells (FIG. 7, panel E). This data indicates that CELA1 expression increases with age and that AAT neutralization of CELA1 is reduced by smoking. Both age and smoking are risk factors for COPD and emphysema.

Binding of CELA1 to Human Lung Tissue is Enhanced by Stretch

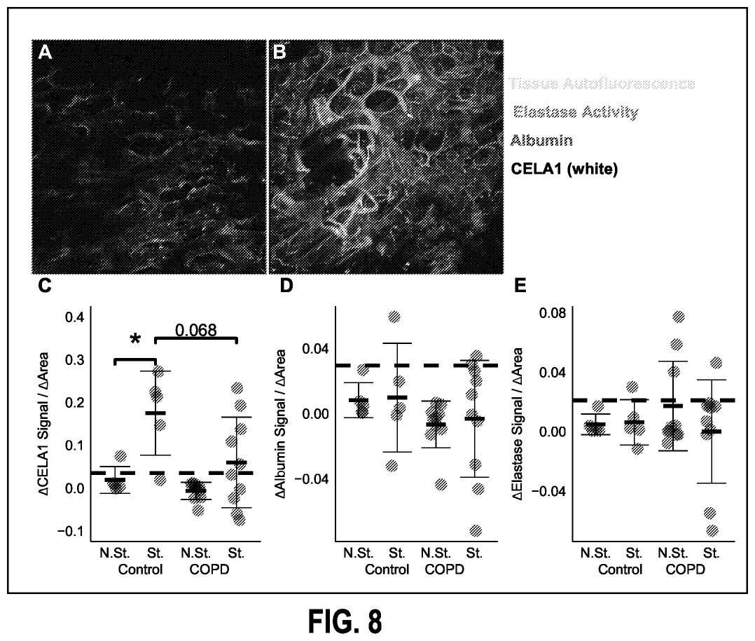

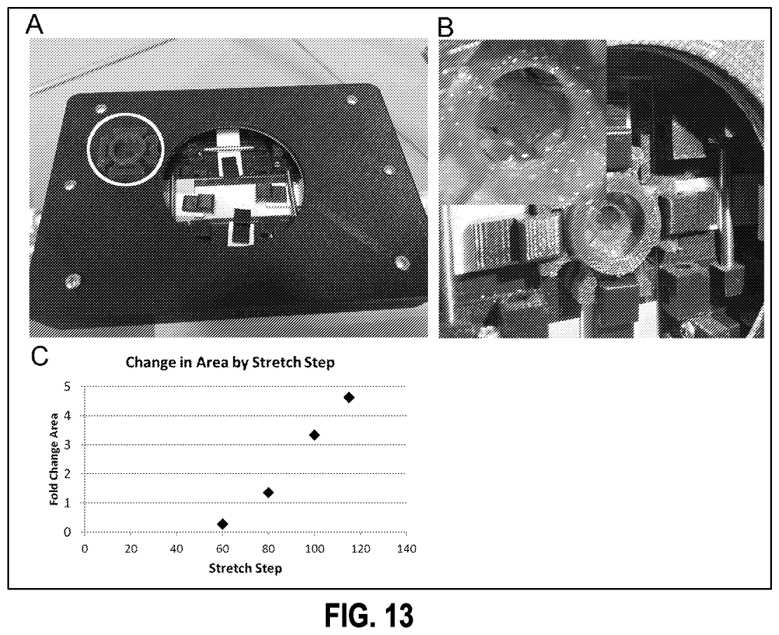

Applicant previously reported a stretch-inducible elastase activity in freshly sectioned mouse lungs,¹⁷ that Cela1 protein bound to these areas of activity,¹⁸ and that Cela1-deficient lung lacked stretch-inducible lung elastase, activity.⁷ Archived lungs from non-lung organ donors and patients with COPD were analyzed for stretch-inducible lung elastase activity using a fluorescent elastin in situ zymography substrate and for quantification of recombinant CELA1 and bovine serum albumin using a 3D-printed biaxial stretching device (FIG. 13). Elastase activity, albumin binding (control) and CELA1 binding signals were normalized to tissue autofluorescence and measured as change in normalized signal divided by bidirectional strain or the time equivalent for unstretched sections (i.e. slope of a plot of signal vs strain for each section. Applicant identified no evidence of stretch-inducible lung elastase activity or increased biding of albumin to lung tissue with stretch, but there was a significant increase in the binding of CELA1 to lung tissue with stretch (FIG. 8). CELA1 binding to human lung tissue is enhanced with stretch although this binding is less in COPD than healthy lung perhaps owing to fibrotic changes and/or altered stretch mechanics in end-stage COPD lung. This data in combination with Applicant's work showing that hydroxyproline cross links inhibit CELA1-mediated elastolysis⁷ suggest that stretching enhances the accessibility of lung elastin fibers to CELA1.

Discussion

It is believed that this disclosure is the first to report a role for CELA1 in lung matrix remodeling in non-AAT-deficient emphysema. Based on Applicant's findings in mouse and human lung, Applicant proposes a 3-step mechanism by which CELA1 mediates progressive airspace destruction. First, CELA1 must be expressed. The increased expression of CELA1 with age could be an important factor in both age-related airspace simplification and the increasing risk of developing COPD with aging. Second, there must be reduced neutralization of CELA1 by AAT. The reduction in AAT-bound CELA1 in aged smoker lung suggest that this might be an important mechanism for emphysema development among smokers. Third, disruption of normal lung architecture increases regional strain, enhances binding of CELA1 to lung elastin and causes additional tissue destruction. This three-step mechanism is consistent with the known COPD risk factors of age, smoking, and preceding lung injury and also explains why individuals with emphysema can experience disease progression despite smoking cessation. Applicant is unaware of any other protease or anti-protease with expression levels that change with age. AAT levels are relatively stable throughout life,¹⁹ and to the best of Applicant's knowledge, age-related changes in other emphysema-associated proteases have not been studied.

AAT neutralizes a host of serine proteases.¹⁹ Applicant previously showed that Cela1 deficient mice were protected from emphysema in an anti-sense oligonucleotide model of AAT deficiency,⁷ and Applicant's associative data in humans suggests that reduced AAT levels in smokers could be a risk factor for increased CELA1 proteolytic activity. While multiple studies have shown that mutations in AAT strongly predispose to emphysema even in the absence of AAT deficiency,²⁰ no such correlative data exists for CELA1. Given the rarity of CELA1 loss of function alleles (the most common has an allele frequency 0.03% and thus homozygous loss of function frequency would be predicted to be 0.09 per 1,000,000), it is not surprising the CELA1 should not have been identified in COPD GWAS studies. It is unclear why CELA1 should be so highly conserved since Cela1^{-/-} mice are viable, fertile, and born at expected Mendelian ratios.⁷

The binding of CELA1 to lung matrix with strain is consistent with previous reports that pancreatic elastase binds lung elastin fibers as a vector of strain.²¹ Elastin degradomic data demonstrated that elastin cross-linking domains retarded CELA1-mediated elastolysis⁷ suggesting that mechanical perturbation of elastin fibers permits CELA1 access to previously hidden sites. The levels of strain used in Applicant's study likely exceed that in human lung physiology, but the time course of the experiment was also necessarily shorter than the years over which emphysema progresses in humans Such dynamic studies of ex vivo human lung are a novel approach to understanding human lung physiology.

Applicant's CELA1 inhibition experiment demonstrates that anti-CELA1 therapies may be used to reduce the proteolytic activity of emphysematous lung. Specimens with higher levels of CELA1 mRNA had a greater reduction in lung elastolytic activity when incubated with control rabbit serum. This likely represented greater neutralization of CELA1 by AAT in the rabbit serum. However, these same specimens had additional lung elastase inhibition after incubation with serum from rabbits immunized with CELA1 peptide while CELA1 mRNA-low specimens did not have additional inhibition. Furthermore, specimens with the highest CELA1 mRNA levels had proportionately more inhibition when incubated with post-immunization serum. Using mouse monoclonal antibodies, Applicant identified four hybridoma clones producing antibodies that inhibit the majority of human lung elastolytic activity. Thus, anti-CELA1 therapies may be used to slow or halt progressive emphysema in established disease.

Methods

Animal Use

Animal Housing

Animal use was approved by the CCHMC Institutional Animal Use and Care Committee (2017-0064). Mice were housed in a pathogen-free facility with 12-hour light/dark cycles and provided chow and water ad lib. Previously generated Cela1^{-/-7} and wild type mice were all on the C57BL/6 background and derived from Applicant's existing colony.

Porcine Pancreatic Elastase Model of Emphysema

A single dose of 2 units porcine pancreatic elastase (PPE, Sigma, St. Louis, Mo.) at a concentration of 10 units/mL diluted in PBS was administered by tracheal instillation as previously described²² to 8-12 week old C57BL/6 mice anesthetized with isoflurane and tracheally cannulated using an 18 gauge angiocatheter.

Aged Mouse Model of Emphysema

WT and Cela1^{-/-} mice were collected at age 70-75 weeks for evaluation of age-dependent alveolar simplification.

Mouse Lung Tissue Collection and Processing

At predetermined time points, mice were anesthetized with 0.2 mL of ketamine/xylazine/acepromazine and sacrificed by exsanguination. The left lung was ligated before inflation and used for protein and RNA analysis, and the right lung inflated at 25 cm H2O water pressure with 4% PFA in PBS, fixed, paraffinized, lobes as previously described (?), and 5 μ m sections created.

Mouse Lung Morphometry

Using the methods of Dunnill²³ on five images from each right lung lobe, mean linear intercepts were determined and used for comparisons.

Anti-CELA1 Antibody Generation

100 micrograms of GEHNLSQNDGTEQYVNVQKIVSHPY (SEQ ID NO: 1) (Genscript, Piscataway, N.J.) peptide in 1 mL of PBS and 1 mL of Freuds complete adjuvant was administered subcutaneously at multiple sites to a New Zealand female rabbit with a subsequent administration of 100 and then 50 micrograms in incomplete Freuds adjuvant on days 21 and 42. Titers were determined by direct ELISA using CELA1-coated plates and titers of <1:5000 considered positive. 7 mL/kg of blood was collected every 2 weeks by marginal vein catheterization using isoflurane anesthesia.

Anti-CELA1 Monoclonal Antibody Generation

CD-1 female mice were immunized with CELA1 peptides (see below) conjugated to CRM197 (a genetically detoxified diphtheria toxin, is widely used as a carrier protein in conjugate vaccines). Each mouse received a primary, subcutaneous immunization of 20 ug of the conjugate with 50% Titermax Gold adjuvant. The mice received subsequent immunizations of 20 ug (day 21) and 10 ug (day 35). Following the day 35 immunization serum samples were obtained and tested for reactivity with CELA1. Based on the results of the serum titration, one mouse was selected for hybridoma production. The mouse received an intravenous immunization of 2 ug of conjugate and three days later the mouse was euthanized and the spleen excised for hybridoma formation with SP2/0.

Supernatants from the resulting hybridomas were tested for reactivity with CELA1 and culture designated 17-103KF4 was subsequently expanded and cloned. Antibodies from the cloned culture, derived from serum-free medium, was used in the studies disclosed herein. These antibodies include AC5, AC7, BA8, CA10, CB4, DG5, DH1, EH2, FG2, HB8, HE5, HF5, HF8, HH1, JA11, JB11, JG6, JG7, KF4 (corresponding to hCELA1 (62-86)), and LD5.

hCELA (30-54)
(SEQ ID NO: 2)
CGTEAGRNSWPSQISLQYRSGGSRYH (“Pep-1”)
hCELA1 (62-86)
(SEQ ID NO: 3)
CRQNWVMTAAHCVDYQKTFRVVAGDH (“Pep-2”)
hCELA1 (104-134)
(SEQ ID NO: 4)
CVVHPYWNSDNVAAGYDIALLRLAQSVTLNSY (“Pep-3”)
hCELA1 (159-183)
(SEQ ID NO: 5)
CGKTKTNGQLAQTLQQAYLPSVDYAI (“Pep-4”)
hCELA1 (220-244)
(SEQ ID NO: 6)
CLVNGKYSVHGVTSFVSSRGCNVSR (“Pep-5”)

Human Lung Tissue Use

Human tissue utilized under a waiver from the CCHMC IRB (2016-9641). Emphysematous lung explants from individuals with COPD and from aged individuals with no documented lung disease were obtained from the NHLBI lung tissue research consortium (LTRC). “Healthy” lung specimens from non-lung organ donors were obtained from National Jewish Health Human Lung Tissue Consortium in Denver, Colo. Flash frozen lung specimens of COPD, non-lung organ donors, and aged lung without known lung disease were obtained from the NIH lung tissue consortium and the National Jewish Health Human Lung Tissue Consortium. Portions of specimens were fixed and sectioned and other portions used for biochemical assays.

Biochemical Assays

Left mouse lungs were ligated and collected prior to inflation and fixation of the right lungs. Protein and RNA was extracted from these specimens and used for Western Blot and PCR. Protein and RNA was similarly extracted from human lung specimens but additionally homogenized lung specimens were analyzed using Enzchek elastase, gelatinase, and proteinase assays (Thermo Fisher E12056, E12055, E6639).

Enzymatic Assays

Frozen human lung specimens were homogenized in RIPA buffer and protein content quantified. Ten μg of protein was used for Enzcheck elastase, gelatinase, and proteinase assays per manufacturer instructions using a Molecular Devices Spectramax M2 plate reader using 4 hour readings for comparisons.

Proximity Ligation In Situ Hybridization (PLISH)

Using previously published methods,²⁴ PLISH for mouse Cela1 human CELA1 mRNA was performed. Briefly, sections were incubated with right and left sided oligonucleotides, then linking oligonucleotides, ligation performed with T4 DNA ligase (New England Biolabs, M0202L). The sequences amplified by rolling circle amplification using Phi29 polymerase (New England Biolabs M0269L), and these oligos detected using a detection oligonucleotide. DAPI counterstaining was performed and sections imaged on a Nikon NiE microscope.

Immunofluorescence

Human lung sections were incubated overnight with 1:500 dilution rabbit anti-CELA1 and 1:500 guinea pig anti-SCGB1A1 antibodies (gift of Jeffrey Whitsett) in 5% donkey serum with subsequent incubation with 1:5000 fluorophore-conjugated secondary antibodies, counterstained with DAPI, and mounted in prolong gold. Images were obtained using a Nikon NiE microscope.

Immunohistochemistry

Human lung sections were immunostained for CELA1 using anti-CELA1 guinea pig antibody⁷ with secondary alone control using the ABC Vectastain kit (Vector Labs, Burlingame, Calif.). 4× tile scanned and 20× images were obtained using a Nikon 90i inverted microscope. Using Nikon elements software, the number of Cela1-positive cells per lung section were morphometrically determined in 4× tile scanned sections.

Western Blot

Mouse lung homogenates were electrophoretically separated, transferred to PVDF membranes, and total protein quantified using a total protein stain (LICOR, 926-11011). Blots were immunostained with anti-CELA1 guinea pig antibody (1:5,000 dilution) and anti-tropoelastin antibody (ab21600, Abcam, 1:5,000 dilution), anti-p16INK (Sigma SAB45000-72, 1:500), p19ARF antibody (Novus Biologics, NB200-169, 1:500), anti-p21 antibody (Novus Biologics, NBP2-29463, 1:500), anti-p53 antibody (Abcam, ab131442, 1:500) and evaluated by densitometry using an Odyssey system (LI-COR Biotechnology, Lincoln, Nebr.). Fold-change values from total protein (ReVERT 700 Total Protein Stain, LI-COR Biotechnology) normalized values was used for calculations.

PCR

RNA was extracted from lung homogenates using RNEasy Mini columns (Qiagen, Valencia, Calif.) and cDNA library synthesized using a High Capacity cDNA Reverse Transcriptase Kit (Applied Biosystems, Foster City, Calif.). For human specimens, Taqman PCR was performed using the primers listed in Table 1 and a QuantStudio6 device (all Applied Biosystems). For mouse specimens Sybr Green PCR was performed using the primers in Table 2 and PowerUp SYBR Green (Applied Biosystems, AB25780).

TABLE 1
TaqMan Primers

| Target | Primer Catalog Number |
|---|---|
| Human Matrix Metaloproteinase-2 | 4331182_Hs01548727 |
| Human Matrix Metaloproteinase-8 | 4453320-Hs01029057 |
| Human Matrix Metaloproteinase-9 | 4453320-Hs00957562_ml |
| Human Matrix Metaloproteinase-12 | 4448892-Hs00159178 |
| Human Matrix Metaloproteinase-14 | 4448892-Hs01037003 |
| Human Proteinase-3 | 4331182-Mm00478323_ml |
| Human Cathepsin G | 4331182-Mm00456011_ml |
| Human Neutrophil Elastase | 4331182-Hs00236952_ml |
| Human Chymotrypsin-like Elastase 1 | 4331182-Hs00608115_ml |
| Eukaryotic 18S RNA | 4333760T |

TABLE 2
Sybr Green PCR Primers

| | | |
|---|---|---|
| MsCela1 FL1-Fwd | TTGTCGGAGAGCACAACCTG | (SEQ ID NO: 7) |
| MsCela1 FL1-Rev | CCAAGACACCAGCAGCATTC | (SEQ ID NO: 8) |
| MsGapdh (66-323) | AGAGTGTTTCCTCGTCCCGT | (SEQ ID NO: 9) |
| mRNA-F | | |
| MsGapdh (66-323) | TGATGTTAGTGGGGTCTCGC | (SEQ ID NO: 10) |
| mRNA-R | | |

Human Ex Vivo Lung Stretch

Ten mm cores of frozen human lung tissue were created using a coring device and hand-cut 100-200 μ m sections were cut using a scalpel on dry ice. These sections were mounted onto a silicone mold and then subjected to biaxial stretch or imaged sequentially over time using a previously published 3D-printed confocal microscope-compatible stretching device^{7,17,18} with Enzchek elastin zymography substrate (10 μg/mL), Texas-Red conjugated albumin (Thermo Fisher A23017, 1 μg/mL) and AF647-conjugated CELA1 (1 μg/mL).⁷ A 100 μm Z-stack at 10× magnification was obtained using a Nikon A1 confocal microscope and the signal for each analyte at sequential levels of biaxial stretch were quantified and normalized to tissue autofluorescence. The rate of elastase activity, albumin binding, and CELA1 binding per fold change in area or time equivalent was measured per section and sections assayed in triplicate. The average rate of change per specimen was used for comparisons.

Human Genomic Studies

CELA1 gene variants were downloaded from the Broad Institute,⁷ dbSNP,¹⁴ and Ensembl.¹⁵ Functional significance of variants was predicted using SIFT and PolyPhen-2.^{16,25}

Statistical Methods

Using R version 3.5.3, the following packages were used for statistical comparisons and graphics generation: ggplot2, gridExtra, cowplot, ggplotify, corrplot, and ggsignif. Welch's t-test and Welch's t-test and Wilcoxon rank-sum test were used to compare parametric and non-parametric data respectively. Parametric data is displayed as line and whisker plots with center line representing mean and whiskers standard deviation. Non-parametric data is displayed as violin plots with center diamond representing median value. For both plot types dots represent individual data points. Correlative analyses were made by Pearson correlation coefficient. For all analyses p-values of less than 0.05 were considered significant.

Aged Mouse Lung Data

There was no difference in Cela1 protein levels in aged vs young WT mice (FIG. S2A). Quantitative morphometry of Hart stained WT and Cela1^{-/-} lung specimens showed a trend towards more insoluble elastin in Cela1^{-/-} lungs (FIG. S2A). There were no statistically significant differences in the amount of senescence-related proteins in aged WT and Cela1^{-/-} lung.

CELA1 in Alveolar Type 2 Cells

Applicant previously demonstrated that in AAT-deficient emphysema, CELA1 mRNA was present in a subset of alveolar type 2 (AT2) cells.⁷ In normal human lung and in COPD, Applicant also found CELA1 protein in AT2 cells (FIG. 11).

Aged Human Lung Supplemental Data

Lung Stretching Data

Image of 3D-printed confocal microscope stretching device (FIG. S4A&B). Quantification of biaxial strain applied by device (FIG. S6C).

Human Genomics Data

Mutations in the CELA1 human genome were assessed for functional significance. Although not predicted to be functional by SIFT, rs74336876 confers a mutation at the CELA1 pro-peptide cleavage site, The unannotated mutation at chr12:51733775 is the most common predicted functional mutation. Both had allele frequencies of <1%.

TABLE 3
Ten Most Common CELA1 Mutations and Functional
Predictions

| | | Transcript | | Functional | Allele |
|---|---|---|---|---|---|
| Position | RSID | Consequence | Annotation | Prediction | Frequency |
| 51737562 | rs17860300 | c.175A > G | missense | Benign | 0.2376 |
| 51723598 | rs60311818 | c.628dupC | frameshift | Benign | 0.2295 |
| 51737607 | rs17860299 | c.130C > T | missense | Benign | 0.1982 |
| 51739648 | rs17860287 | c.30G > C | missense | Benign | 0.09864 |
| 51723499 | rs17860364 | c.728A > G | missense | Benign | 0.09134 |
| 51739640 | — | c.38C > T | missense | Benign | 0.00909 |
| 51739625 | rs74336876 | c.53G > A | missense | Benign | 0.007377 |
| 51735144 | rs17860313 | c.347G > A | missense | Benign | 0.001723 |
| 51735071 | — | c.419delC | frameshift | Benign | 0.0005204 |
| 51733775 | — | c.478G > A | missense | Loss-of- | 0.0002924 |
| | | | | Function | |

Animal Use

Animal use was approved by the CCHMC Institutional Animal Use and Care Committee (2017-0064). Using the methods of Dunnill,²³ mean linear intercepts in lungs of 8-12 week-old

Cela1^{-/-7} and WT mice on the C57BL/6 background in the tracheal porcine pancreatic elastase (PPE) model²² and untreated mice aged 70-75 weeks were determined. Anti-CELA1 polyclonal antibody was generated using a single female New Zealand rabbit using the same peptide and methods as reported for guinea pig.¹⁷

Human Lung Tissue

Human tissue utilized under a waiver from the CCHMC IRB (2016-9641). COPD and aged human lung specimens were obtained from the NHLBI Lung Tissue Consortium and control and COPD specimens from the National Jewish Health Human Lung Tissue Consortium.

Enzymatic Assays

Human lung protease, gelatinase, and elastase activity was quantified using commercial fluorometric assays.

Proximity Ligation In Situ Hybridization (PLISH) and Immunofluorescence

Using oligos, (see previously published methods)²⁴ CELA1 mRNA was visualized with immunofluorescent co-staining using antibodies outlined in the supplement.

PCR

Taqman and Sybr Green PCR was used for mouse and human lung PCR using primers in Tables 1 & 2.

Ex Vivo Human Lung Stretch

Using a previously described lung stretching technique and device,^{7,17,18} the stretch-dependent binding of recombinant CELA1, albumin, and elastolytic activity of frozen human lung sections was determined.

### REFERENCES

1. Bhandari, A. & McGrath-Morrow, S. Long-term pulmonary outcomes of patients with bronchopulmonary dysplasia. Seminars in perinatology 37, 132-7 (2013).

2. Brantly, M. et al. The Diagnosis and Management of Alpha-1 Antitrypsin Deficiency in the Adult. Chronic Obstructive Pulmonary Diseases (Miami, Fla.) 3, 668-682 (2016).

3. Campbell, E., Pierce, J., Endicott, S. & Shapiro, S. Evaluation of extracellular matrix turnover. Methods and results for normal human lung parenchymal elastin. Chest 99, 49S (1991).

4. Cosio, M. G. et al. Alpha-1 Antitrypsin Deficiency: Beyond the Protease/Antiprotease Paradigm. Annals of the American Thoracic Society 13 Suppl 4, S305-10 (2016).

5. Gotzsche, P. C. & Johansen, H. K. Intravenous alpha-1 antitrypsin augmentation therapy for treating patients with alpha-1 antitrypsin deficiency and lung disease. The Cochrane database of systematic reviews 9, Cd007851 (2016).

6. Liu, S., Young, S. M. & Varisco, B. M. Dynamic expression of chymotrypsin-like elastase 1 over the course of murine lung development. American journal of physiology. Lung cellular and molecular physiology 306, L1104-16 (2014).

7. Joshi, R. et al. Role for Cela1 in Postnatal Lung Remodeling and AAT-deficient Emphysema. American journal of respiratory cell and molecular biology (2018) doi:10.1165/rcmb.2017-0361OC.

8. Pugh, M. E. & Hemnes, A. R. Development of pulmonary arterial hypertension in women: interplay of sex hormones and pulmonary vascular disease. Womens Health (Lond Engl) 6, 285-96 (2010).

9. He, S. & Sharpless, N. E. Senescence in Health and Disease. Cell 169, 1000-1011 (2017).

10. Kusko, R. L. et al. Integrated Genomics Reveals Convergent Transcriptomic Networks Underlying COPD and IPF. American Journal of Respiratory and Critical Care Medicine (2016) doi:10.1164/rccm.201510-20260C.

11. Keene, J. D. et al. Biomarkers Predictive of Exacerbations in the SPIROMICS and COPDGene Cohorts. American journal of respiratory and critical care medicine 195, 473-481 (2017).

12. Woodruff, P. G. et al. Clinical Significance of Symptoms in Smokers with Preserved Pulmonary Function. N. Engl. J. Med. 374, 1811-1821 (2016).

13. 1000 Genomes|A Deep Catalog of Human Genetic Variation. https://www.internationalgenome.org/.

14. Home-dbGaP-NCBI. https://www.ncbi.nlm nih gov/gap/.

15. Landrum, M. J. et al. ClinVar: public archive of relationships among sequence variation and human phenotype. Nucleic Acids Res 42, D980-D985 (2014).

16. Adzhubei, I., Jordan, D. M. & Sunyaev, S. R. Predicting functional effect of human missense mutations using PolyPhen-2. Curr Protoc Hum Genet Chapter 7, Unit7.20-Unit7.20 (2013).

17. Young, S. M. et al. Localization and stretch-dependence of lung elastase activity in development and compensatory growth. Journal of applied physiology (Bethesda, Md.: 1985) 118, 921-31 (2015).

18. Joshi, R. et al. Stretch regulates expression and binding of chymotrypsin-like elastase 1 in the postnatal lung. FASEB journal : official publication of the Federation of American Societies for Experimental Biology 30, 590-600 (2016).

19. Janciauskiene, S. & Welte, T. Well-Known and Less Well-Known Functions of Alpha-1 Antitrypsin. Its Role in Chronic Obstructive Pulmonary Disease and Other Disease Developments. Annals of the American Thoracic Society 13 Suppl 4, S280-8 (2016).

20. Busch, R. et al. Genetic Association and Risk Scores in a COPD Meta-Analysis of 16,707 Subjects. American journal of respiratory cell and molecular biology (2017) doi:10.1165/rcmb.2016-0331OC.

21. Jesudason, R. et al. Mechanical forces regulate elastase activity and binding site availability in lung elastin. Biophysical journal 99, 3076-83 (2010).

22. Suki, B., Bartolak-Suki, E. & Rocco, P. R. M. Elastase-Induced Lung Emphysema Models in Mice. Methods Mol Biol. 1639, (2017).

23. Dunnill, M. S. Quantative Methods in the Study of Pulmonary Pathology. Thorax 17, 320-328 (1962).

24. Nagendran, M., Riordan, D. P., Harbury, P. B. & Desai, T. J. Automated cell type classification in intact tissues by single-cell molecular profiling. eLife in revision, (2017).

25. Sim, N.-L. et al. SIFT web server: predicting effects of amino acid substitutions on proteins. Nucleic Acids Res 40, W452-W457 (2012).

All percentages and ratios are calculated by weight unless otherwise indicated.

All percentages and ratios are calculated based on the total composition unless otherwise indicated.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “20 mm” is intended to mean “about 20 mm.”

Every document cited herein, including any cross referenced or related patent or application, is hereby incorporated herein by reference in its entirety unless expressly excluded or otherwise limited. All accessioned information (e.g., as identified by PUBMED, PUBCHEM, NCBI, UNIPROT, or EBI accession numbers) and publications in their entireties are incorporated into this disclosure by reference in order to more fully describe the state of the art as known to those skilled therein as of the date of this disclosure. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications may be made without departing from the spirit and scope of the invention. It is therefore intended to cover in the appended claims all such changes and modifications that are within the scope of this invention.

1. A method of treating a lung disease in a human subject, comprising administering a CELA1 inhibitor to a human subject in need thereof.

2. The method of claim 1 wherein said lung disease is selected from chronic obstructive pulmonary disease (COPD), optionally COPD GOLD Stage I or greater, emphysema, optionally wherein said emphysema is in an individual having genetic AAT deficiency, optionally wherein said emphysema is CT confirmed emphysema, optionally wherein said emphysema is progressive emphysema, progressive airspace destruction after injury, and combinations thereof.

3. The method of claim 1 wherein said CELA1 inhibitor is an antigen binding protein (ABP).

4. The method of claim 1, wherein said CELA1 inhibitor is an antigen binding protein (ABP) which binds to one or more residues, or at least two residues, or at least three residues, or at least four residues, or at least five residues, or at least six residues, or at least seven residues, or at least eight residues, or at least nine residues, or at least ten residues in a peptide selected from SEQ ID NO: 2, SEQ ID NO: 3, SEQ ID NO: 4, SEQ ID NO: 5, or SEQ ID NO: 6, preferably wherein said ABP is an antibody, preferably wherein said ABP an isolated monoclonal antibody, preferably wherein said antibody is a human antibody.

5. The method of claim 1, wherein said CELA1 inhibitor is an antisense oligonucleotide (ASO).

6. The method of claim 1, wherein said anti-CELA1 inhibitor inhibits at least 50%, or at least 60%, or at least 70%, or at least 80%, or at least 90%, or about 100%, of human lung elastolytic activity.

7. The method of claim 4, wherein said anti-CELA1 inhibitor is an antibody comprising an arginine-glycine-aspartic acid (RGD) motif

8. The method of claim 1 wherein said CELA1 inhibitor is administered intravenously.

9. The method of claim 1 wherein said CELA1 inhibitor is administered via a nebulizer.

10. The method of claim 1, wherein said administration is selected from daily, every other day, weekly, every other week, every three weeks, or monthly.

11. A composition comprising an antigen binding protein (ABP), preferably an isolated monoclonal antibody that binds to human CELA1 at least one residue, or at least two residues, or at least 3 residues, or at least 4 residues, or at least 5 residues, or at least 6 residues, or at least 7 residues of, or at least 8 residues of, or at least 9 residues of, or at least 10 residues, or at least 11 residues, or at least 12 residues of, or at least 13 residues, or at least 14 residues of, or at least 15 residues of, or at least 16 residues of, or at least 17 residues of, or at least 18 residues of, or at least 19 residues of, or at least 20 residues of, or at least 21 residues of, or at least 22 residues of, or at least 23 residues of or at least 24 residues of, or at least 25 residues of, or each residue of a sequence selected from SEQ ID NO: 2, SEQ ID NO: 3, SEQ ID NO: 4, SEQ ID NO: 5, or SEQ ID NO: 6.

12. The composition of claim 11 wherein said monoclonal antibody is a human antibody.

13. The composition of claim 11 further comprising a carrier that is one or both of sterile and isotonic.

14. A single-chain variable fragment (scFv) comprising a variable region of the heavy (VH) of the antibody of claim 11 and a variable region of the light chain (VL) of the antibody of claim 11, wherein said VH and said VL are connected by a linker peptide, and wherein said scFv is specific for at least one region of human CELA1.

15. The isolated monoclonal antibody of claim 11, wherein said antibody inhibits at least 50%, or at least 60%, or at least 70%, or at least 80%, or at least 90%, or about 100%, of human lung elastolytic activity.

16. The scFv of claim 14, wherein said scFv inhibits at least 50%, or at least 60%, or at least 70%, or at least 80%, or at least 90%, or about 100%, of human lung elastolytic activity.
